(19) United States
(12) Patent Application Publication
(10) Pub. No.: US 2008/0102465 A1
(43) Pub. Date: May 1, 2008

(54) PTHRP-BASED PREDICTION AND DIAGNOSIS OF BONE DISEASE

(75) Inventors: Andrew C. KARAPLIS, Kirkland (CA); David GOLTZMAN, Westmount (CA)

Correspondence Address: OGILVY RENAULT LLP, 1981 MCGILL COLLEGE AVENUE, SUITE 1600, MONTREAL, QC, H3A2Y3, CA

(73) Assignee: MCGILL UNIVERSITY, Montreal (CA)

(21) Appl. No.: 11/869,199

(22) Filed: Oct. 9, 2007

Related U.S. Application Data
(63) Continuation of application No. 10/954,220, filed on Oct. 1, 2004, now abandoned.
Application No. 10/954,220 is a continuation of application No. 10/448,117, filed on May 30, 2003, now abandoned.
(60) Provisional application No. 60/384,122, filed on May 31, 2002.

(51) Int. Cl. C12Q 1/68

(52) U.S. Cl. 435/6

(57) ABSTRACT

The invention provides methods of diagnosing bone disease and/or a susceptibility thereto, in an individual. The method includes screening a biological sample obtained from the individual for one or more genetic indicators of bone disease in said PTHrP gene of the individual, and diagnosing the individual based on a characterization of the genetic indictor(s) detected. A genetic indicator of the invention preferably includes a genetic segment of a PTHrP gene. More preferably, a genetic segment of a PTHrP gene includes a VNTR containing region. The invention further relates to transgenic non-human mammals for the study of bone disease and/or bone conditions or for drug discovery, lead optimization, identification of drug candidates & drug development, wherein a transgenic mammal of the invention may be (a) homozygous for disrupted PTHrP gene only in osteoblast cells of said mammal (PTHrPflox/flox crecol I); (b) heterozygous for disrupted PTHrP gene (PTHrP^{-/+}) in all cells of said mammal; or (c) heterozygous for disrupted PTHrP gene (PTHrP^{-/+}) only in osteoblast cells of said mammal.

### CROSS-REFERENCE TO RELATED APPLICATIONS

This application is a continuation-in-part of and claims priority from U.S. application Ser. No. 10/954,220 filed Oct. 1, 2004, published as US No. 2005/0089909 on Apr. 28, 2005, which is a continuation-in-part of and claims priority from U.S. application Ser. No. 10/488,117 filed May 30, 2003, published as US No. 2004/0005619 on Jan. 8, 2004, which claims the benefit of priority of U.S. Provisional Patent Application No. 60/384,122 filed May 31, 2002, each of which are incorporated herein, in their entirety, by reference.

### TECHNICAL FIELD

The invention relates to methods and materials for the prediction, diagnosis and treatment of disease. More particularly, the present invention relates to methods and materials for the prediction, diagnosis and treatment of bone disease(s). The present invention also describes systems and methods for screening and detecting compounds that are potentially therapeutic or prophylactic in the treatment of bone disease(s), and in particular, osteoporosis.

### BACKGROUND OF THE INVENTION

Bone diseases affect women, men, and children of all ages. From infancy to old age, bone disease profoundly alters the quality of life for millions of North Americans. Each year, osteoporosis, Paget's disease, osteogenesis imperfecta and multiple myeloma, among other bone diseases, strike more than 30 million people in the USA alone and cause loss of independence, disability, pain, and death. The annual cost of acute and long-term care relating to bone diseases in the United States is estimated to be $20 billion. As the population ages, these costs are expected to increase to more than $60 to $80 billion by the year 2020. Without intervention, including improved methods of diagnosis, especially pre-onset prognostic tests and potential prophylactic treatments, chronic diseases, such as osteoporosis, will drive up the cost of acute and long-term care well into the next century and overwhelm any effort to contain health care costs.

Osteoporosis and related fractures arising from diminished bone density are particularly common in older individuals and contribute substantially to the healthcare costs and burden of illness associated with the disease. Although osteoporosis has many causes, about 80% of the underlying etiology is genetic. Unfortunately, there are no tests commercially available currently that can determine an individual's predisposition for osteoporosis prior to the disease onset. Very often, an individual is diagnosed with osteoporosis only after the disease has progressed extensively. Failure to provide early detection of bone disease and/or a predisposition for bone disease drives up the cost and suffering associated with such a disease.

Recently, studies in the laboratories of the instant applicants and others, have provided compelling evidence that a protein expressed in osteoblasts, the bone-forming cells of the skeleton, namely the parathyroid hormone-related protein, PTHrP, is critical for the proper recruitment, proliferation, differentiation, and function of these cells, wherein these processes are pivotal for maintenance of proper bone density and preventing the development of osteoporosis (Horwitz et al. J Clin Endocrinol Meta., February 2003, 88(2), 569-575) which are herein incorporated by reference.

Summarized below are some details and evidence generated by the applicants of the present invention:

Mice homozygous for PTHrP gene inactivation, (i.e. PTHrP^{-/-} mice) (Karaplis et al., (1994) Genes Dev. 8, 277-289; Amizuka et al., (1994) J. Cell Biol. 126, 1611-1623) were observed to possess dyschondroplastic skeletal abnormalities and altered endochondral bone formation, that culminate in their death in the immediate peripartum period. Therefore, since the PTHrP^{-/-} mice are knockout mice missing both copies of the PTHrP gene from their genome, wherein PTHrP is absent from all cells, such homozygous PTHrP^{-/-} mice are not viable.

On the other hand, mice heterozygous for PTHrP gene inactivation, i.e. PTHrP^{-/+} mice, (knockout mice missing one copy of the PTHrP gene from genome when normally there are two copies) are phenotypically normal at birth, but develop, by about 3 months of age, features consistent with premature osteoporosis (Amizuka et al., (1996) Developmental Biol. 175, 166-176). This severe form of osteoporosis is associated with decreased PTHrP expression in the skeleton and characterized by a marked decrease in trabecular bone volume and connectivity (decreased degree of anisotropy and increased structure model index), observations which are very characteristic and representative of the human form of osteoporosis.

It is well known in the prior art that there are various compounds that have been shown to act as anabolic agents with respect to bone disease. For example, bone anabolic agents, such as PTH (1-34); PTH (1-84), and PTHrP (1-36), have been shown clinically to be anabolic agents. As will be discussed below, PTH (1-34) is a confirmed anabolic agent which is analogous to PTH (1-84) and PTHrP (1-36) in terms of its interactions with the PTHR1 receptor on the osteoblast surface and action as a bone anabolic agent, was used to support and provide experimental evidence for the teachings of the present invention.

As such, it has been demonstrated that the level of PTHrP correlates with osteoporotic bone disease. However, there remains a need for improved methods and systems for characterizing the basis of bone disease, to provide further insight into the mechanism of such diseases, and to develop sensitive diagnostic and treatment methods relating thereto. Furthermore, there remains a need for improved methods for detecting bone disease prior to the onset of the disease and/or providing means to determine a genetic predisposition thereto so as to allow for the implementation of corresponding genotype-specific customized treatment and/or prophylactic regimes to improve the health of an effected individual.

### SUMMARY OF THE INVENTION

An objective of the present invention to provide a method for diagnosing bone disease in an individual.

A further objective of the present invention to provide a method for characterizing a predisposition for, and/or susceptibility to, bone disease in an individual.

A further objective of the present invention to provide a method for selectively treating bone disease and/or a predisposition for bone disease in an individual diagnosed therewith.

It is yet a further object of the present invention to provide a method which enables screening for novel therapeutics for the treatment of bone disease.

In accordance with an embodiment of the present invention, indicators of bone disease have been characterized. More specifically, genetic indicators of bone disease have been characterized within the PTHrP gene. In particular, a genetic indicator of osteoporosis has been characterized within the PTHrP gene in accordance with a preferred embodiment of the present invention. Furthermore, the indicators of the present invention provide novel diagnostic and therapeutic targets for characterizing and/or treating a predisposition for bone disease, such as osteoporosis, for example. In addition, the indicators of bone disease, as identified in accordance with the present invention, provide novel targets for modulating expression of the PTHrP gene in connection with providing a treatment and/or prophylactic regime in an individual in need thereof. As described in accordance with one embodiment of the present invention, indicators of bone disease occur within a variable number of tandem repeat (VNTR) region within an intron of the PTHrP gene. Preferably, the intron is located between exons VI and VII of the PTHrP gene. A PTHrP gene of the present invention preferably refers to a mammalian PTHrP gene. In accordance with one embodiment of the present invention, a PTHrP gene refers to a murine PTHrP gene. More preferably, a PTHrP gene of the present invention refers to a human PTHrP gene.

In accordance with an embodiment of the present invention, a genetic indicator of the present invention is a genetic segment within a VNTR region of the PTHrP gene. Preferably, this genetic segment is characterized as an indicator of bone disease or an indicator of a predisposition for bone disease on the basis of allele length. More preferably, a genetic indicator of the present invention is characterized according to the number of predetermined repeat sequences within the VNTR region of the PTHrP gene. An embodiment of the present invention characterizes a genetic indicator of bone comprising at least one 9-mer nucleotide sequence within the VNTR region of the PTHrP gene. Preferably, a genetic indicator comprises a repeat of two or more 9-mer nucleotide sequences of the present invention.

The term “genetic indicator” is intended to mean a genetic marker within a gene of interest that provides an indication of an individuals genetic predisposition and/or potential to develop a disease, preferably bone disease, in the individual identified therewith. For example, genetic indicators may be markers or polymorphisms within the PTHrP gene, and more preferably, within the VNTR region of the PTHrP gene, wherein said genetic indicators, or markers, may comprise a tandem repeat, such as a 9-mer tandem repeat, wherein variations in the number of tandem repeats would consequently vary the length of the VNTR region, whereby the length of the VNTR region is effectively a genetic indicator that correlates specific allele lengths with specific conditions, such as the correlation of specific VNTR region lengths to the diagnosis or prediction of an individual's susceptibility to bone disease.

That is to say, in accordance with an embodiment of the present invention, genetic indicators within the VNTR region of the PTHrP gene are the allelic variations in the length of the VNTR region. A determination of the allelic length of the VNTR region of the PTHrP gene relates to an allelic polymorph for that sample. The genetic indicator, as determined by the allelic length of the VNTR region, will provide a genetic indication of the risk associated with the susceptibility to bone disease for that patient. The genetic indicator, or genetic marker, or allelic length/will vary depending on number of 9-mer tandem repeats present in the VNTR of the PTHrP gene. In a preferred embodiment of the present invention, the allelic length may comprise a length of 252 bp, 288 bp, 332 bp, 356 bp, 378 bp, 393 bp, 414 bp or 460 bp.

The term “allele” is intended to mean an alternative form of a genetic segment or a region of a gene of interest that provides a genetic indicator in accordance with the present invention. Preferably, an allele of the present invention is a form of the variable number tandem repeat (VNTR) region of the PTHrP gene. For example, a specific VNTR region length may correlate or indicate a specific number of tandem repeats, wherein variations in the number of tandem repeats would in effect vary the length of the VNTR region, such that variations in VNTR length are allelic variations of the VNTR region of a specific gene, more preferably, in accordance with the present invention, in the PTHrP gene. Accordingly, for the purposes of the present disclosure, an allele may refer to a specific VNTR region length, wherein variations in VNTR length are effective allelic determinants of a specific genotype or phenotype relating to the PTHrP gene.

The term “diagnosis” or “diagnosing” refers to the determination or identification of a disease state or a predisposition or susceptibility for developing a disease in a mammal, based on, at least in part, a genetic indication thereof. Accordingly, the term “diagnosis” or “diagnosing” may encompass a prognosis.

The term “bone disease state” refers to a degree of severity of the disease in an afflicted individual.

The term “individual” refers a subject patient or mammal. Preferably, a mammal is a mouse. More preferably, a mammal is a human.

Accordingly, the present invention provides a method of diagnosing an individual for the presence, susceptibility or predisposition to bone disease, said method comprising: a) obtaining a biological sample suitable for detecting a parathyroid hormone related peptide (PTHrP) gene from said individual; b) characterizing allelic polymorphisms in the PTHrP gene by determination of the allelic length of the variable number of tandem repeat (VNTR) region of said PTHrP gene; c) diagnosing said individual based on the results of step (b).

In a preferred embodiment, the present invention provides a method of diagnosing an individual for the presence, susceptibility or predisposition to bone disease, said method comprising: a) obtaining a biological sample suitable for detecting a parathyroid hormone related peptide (PTHrP) gene from said individual; b) screening for one or more genetic indicators of bone disease in said PTHrP gene; c) characterizing the PTHrP gene by determination of the allelic length of the variable number of tandem repeat (VNTR) region of said PTHrP gene based on said genetic indicators; c) diagnosing said individual based on the results of step (b).

In a preferred embodiment, the biological sample is a biological fluid or tissue comprising isolatable genomic DNA; the allelic length of the VNTR region is correlated with the presence, predisposition or susceptibility to bone disease. Bone disease is selected from the group consisting of osteoporosis, osteomalacia, osteopenia, osteopetrosis, Paget's disease and renal osteodystrophy metastatic bone disease. Determining the allelic length of the VNTR region comprises the amplification of said VNTR region using nucleotide probes specific thereto. In a preferred embodiment, the allelic length of the VNTR region varies based on the number of 9-mer tandem repeats comprised therein; wherein the allelic length of the VNTR region is between 252 to 460 base pairs; where the VNTR region comprises allelic lengths of 252, 288, 332, 356, 378, 393, 414 or 460 base pairs. In accordance with the present invention, the 9-mer tandem repeats are selected from the group consisting of GTATATATA (SEQ ID NO: 3) and ATATATATA (SEQ ID NO: 4).

The present invention also provides the use of genetic indicators in the PTHrP gene as allelic determinants of various allelic polymorphs, for the diagnosis of the presence, susceptibility or predisposition to bone disease. The allelic polymorphisms in the PTHrP gene depend on the allelic length of the VNTR region of said PTHrP gene. The allelic length of the VNTR region varies based on the number of 9-mer tandem repeats comprised therein; wherein the 9-mer tandem repeats are selected from the group consisting of GTATATATA and ATATATATA; and the VNTR region comprises allelic lengths of 252, 288, 332, 356, 378, 393, 414 or 460 base pairs.

The present invention also provides a method of treating a patient, having a PTHrP gene, identified a bone disease or a predisposition or susceptibility thereto, said method comprising the administration of a treatment regime based on the allelic characterization of the VNTR region of the PTHrP gene of said patient; wherein the allelic characterization of the VNTR region comprises a determination of the allelic length of said VNTR region. In a preferred embodiment, the treatment regime is a genotype-specific treatment regime, which may be variable over time. In a preferred method of treatment of the present invention, the treatment regime comprises modulating the expression of the PTHrP gene in osteoblast cells; wherein a preferred treatment regime comprises increasing local PTHrP-like activity in osteoblast cells. In an embodiment, the treatment regime comprises the administration of exogenous PTH and/or PTHrP, or analogues thereof, locally to osteoblast cells. In a preferred embodiment, the treatment regime comprises the administration of exogenous PTH (1-34).

The present invention also provides a transgenic non-human mammal homozygous for disrupted PTHrP gene (PTHrP^{-/-}) (PTHrPflox/flox crecol I) in osteoblast cells specifically, wherein, non-osteoblast cells are not disrupted with respect to the PTHrP gene.

The present invention also provides a transgenic non-human mammal heterozygous for disrupted PTHrP gene (PTHrP^{-/+}) in all cells of said mammal, wherein said all cells comprises osteoblast and non-osteoblast cells.

The present invention also provides a transgenic non-human mammal heterozygous for disrupted PTHrP gene (PTHrP^{-/+}) in osteoblast cells specifically, wherein, non-osteoblast cells are not disrupted with respect to the PTHrP gene.

The transgenic non-human mammals of the present invention have been shown to work as an in vivo system which allow for screening and identifying compounds for therapeutic and/or prophylactic properties for bone diseases.

In accordance with the present invention, the somatic cells of the mammals of the present invention comprise osteoblast-specific PTHrP disruption means capable of disrupting a PTHrP gene specifically in osteoblast cells without disrupting the PTHrP gene in non-osteoblast cells. In a preferred embodiment, the osteoblast-specific PTHrP disruption means comprises: a) a Cre-recombinase gene under control of an osteoblast-specific promoter; and b) loxP sites which flank the PTHrP gene or a portion thereof. In an embodiment, the osteoblast-specific promoter is a modified form of the type I collagen promoter. In a preferred embodiment, exon 4 of the PTHrP gene is disrupted in said mammals. In a preferred embodiment, a mammal of the present invention is a mouse.

The present invention additionally provides for the use of a mammal of the present invention, for studying bone development and bone diseases. In a preferred embodiment, the use of the mammal for studying the effects of compounds, agents or drugs for the prevention and treatment of bone diseases is provided. In another embodiment, the use of the mammal for identifying compounds, agents or drugs having therapeutic or prophylactic properties in the prevention and/or treatment of bone diseases and/or delaying the progression thereof is provided. A mammal of the present invention may be used as an in vivo assay system capable of screening and identifying compounds for therapeutic and/or prophylactic properties for bone diseases.

The present invention also provides for the use of a compound selected from the group consisting of PTH (1-34), PTH (1-84), PTHrP (1-36), PTHrP (1-139) their cyclic and non-cyclic analogs, their peptidomimetic analogs, their small molecule drug analogs, and other bone anabolic agents to study the effects of such compounds on bone formation using a non-human mammal of the present invention. Also provided is the use of a compound selected from the group consisting of PTH (1-34), PTH (1-84), PTHrP (1-36), PTHrP (1-139), their cyclic and non-cyclic analogs, their peptidomimetic analogs, their small molecule drug analogs, and other bone anabolic agents to treat, prevent or delay the progression of bone disease.

The present invention also provides a single-stranded nucleic acid having a nucleotide sequence comprising one or more repeats of GTATATATA and/or ATATATATA, wherein said nucleotide sequence has complimentarity to a region of a PTHrP gene. In a preferred embodiment, the single-stranded nucleic acid may be used as a genetic indicator of bone disease and/or a predisposition thereto; wherein the single-stranded nucleic acid sequence is 252, 288, 332, 356, 378, 393, 414 or 460 nucleotides in length.

The present invention provides a single-stranded oligonucleotide comprising nucleotide sequences GTATATATA and/or ATATATATA, or multiple repeats thereof, for use in identifying candidate compounds having the ability to modulate PTHrP expression.

Also provided is a single-stranded oligonucleotide comprising the sequence of SEQ ID NO: 1, or a compliment thereof; wherein an isolated single-stranded oligonucleotide of the present invention hybridizes to a hybridization probe under stringent conditions; wherein the oligonucleotide may be used for identifying protein that modulates PTHrP expression in vivo. The use of the oligonucleotide for identifying candidate compounds having the ability to modulate PTHrP expression is also embodied. The oligonucleotide of the present invention may be used to screen candidate compounds, wherein said oligonucleotide binds said compounds.

SEQ ID NO: 1 is the portion of the VNTR comprising two 9-mer oligo repeat sequences. This sequence potentially binds a protein (potentially a transcription factor) whose cDNA has been shown to be expressed in human mesenchymal stem cells as they are differentiating into osteoblasts. This protein, or the transcription factor, may regulate the PTHrP expression and hence relate critically to bone formation. This sequence could be used as a target to develop novel therapeutics based on a potential transcription factor binding to it. More specifically, the sequence could be used as bait to identify new proteins that bind to it and could regulate PTHrP expression, in fact, this oligo sequence could be bound to solid support in an HPLC column and may be able to allow for the identification of proteins in the osteoblast microenvironment binding to it and use these proteins for therapeutic purposes.

The present invention also provides a commercial package or kit for the diagnosis and/or prediction of bone disease in an individual, said kit comprising: a) detection means capable of characterizing allelic polymorphisms by the screening for genetic indicators in a PTHrP gene; and b) instructions for characterizing said allelic polymorphisms and correlating a diagnosis and/or prediction of bone disease therefrom; wherein the detection means comprises probes capable of hybridizing a tandem repeat of the VNTR region of said PTHrP gene; wherein said allelic polymorphisms comprises variations in the allelic length of the VNTR region of said PTHrP gene; wherein said kit further comprising means for determining the allelic length of the VNTR region of said PTHrP gene; wherein said kit further comprises means for amplifying a VNTR region of said PTHrP gene. In a preferred embodiment, the tandem repeat comprises a sequence of GTATATATA or ATATATATA; the allelic length of the VNTR region comprises 252, 288, 332, 356, 378, 393, 414 or 460 base pairs.

In a preferred embodiment, the present invention provides the use of specific probes that specifically bind to DNA (preferably genomic DNA) in a biological sample, to bind to the VNTR region of the PTHrP gene. In a preferred embodiment, the DNA is amplified, purified and the length is determined (preferably using PAGE, or any standardized protocol). The VNTR region length is polymorphic, wherein, depending on number of 9-mer tandem repeats present in the VNTR of the PTHrP gene, the VNTR region can vary in length (i.e. various allelic lengths). In a preferred embodiment, VNTR allelic lengths can be 252 bp, 288 bp, 332 bp, 356 bp, 378 bp, 393 bp, 414 bp or 460 bp. The VNTR lengths correspond to different allelic variants (polymorphs) of the PTHrP gene, wherein the allelic length determined (using probes specific to the 9-mer repeat present in the VNTR), define specific allelic variants (i.e. different length=different allele of PTHrP), where the allelic length determined provides prognosis of bone disease or the susceptibility thereto.

In accordance with one aspect of the present invention, there is provided a method of diagnosing bone disease and/or a predisposition therefore in an individual, said method comprising: (a) obtaining a biological sample from said individual, said sample suitable for detecting a parathyroid hormone related peptide (PTHrP) gene of said individual therein; (b) screening for one or more genetic indicators of bone disease in the PTHrP gene of said individual; and (c) providing a diagnosis of said individual with respect to bone disease and/or a predisposition therefore based on the results obtained in step (b).

A biological sample of the present invention is preferably a sample comprising DNA, preferably genomic DNA, of said individual and can be any biological sample such as blood, saliva, hair, skin etc. from which DNA can be isolated, whereby the biological sample obtained is suitable for amplifying and/or detecting the DNA sequence contained therein.

One or more genetic indicators of the present invention may include a region within said PTHrP gene. According to an embodiment of the present invention, one or more genetic indicators of the present invention may comprise a variable number of tandem repeat (VNTR) region within said PTHrP gene. Furthermore, the one or more genetic indicators of the present invention may comprise of variable number of tandem repeat (VNTR) region(s) within said PTHrP gene wherein said region(s) is characterized on the basis of allele length. According to a preferred embodiment of the present invention, a genetic indicator is a segment of the variable number tandem repeat (VNTR) region of a PTHrP gene having a predetermined length, wherein the genetic indicator correlates to a bone disease state and/or a susceptibility for bone disease.

In accordance with an embodiment of the present invention, a bone disease is preferably osteoporosis, but may be additionally osteomalacia, osteopenia, osteopetrosis, Paget's disease, renal osteodystrophy, or any degenerative condition relating to bone or cartilage.

The region of interest in a PTHrP gene of the present invention is preferably the VNTR region. More preferably, this region includes a variable number of tandem repeats in the nucleotide sequence thereof. Furthermore, in accordance with embodiments of the present invention, a genetic indicator is preferably a segment of the PTHrP gene within the VNTR region that is between 252 to 460 base pairs in length.

According to a further preferred embodiment of the present invention, a genetic indicator includes a segment within the VNTR region of a PTHrP gene that is 252 base pairs (bp), 288 bp, 332 bp, 35 bp, 378 bp, 393 bp, 414 bp or 460 bp in length. A genetic indicator of the present invention may also comprise at least one nucleotide repeat of a 9-mer nucleotide sequence within the PTHrP gene. More preferably, the 9-mer nucleotide sequence of the present invention may be further referred to herein as a variable number tandem repeat (VNTR) selected from the group consisting of GTATATATA and/or ATATATATA.

In accordance with one aspect of the present invention, there is provided a method of diagnosing a susceptibility for bone disease in an individual; said individual having a PTHrP gene; said method comprising: (a) obtaining a biological sample suitable for screening for one or more genetic indicators of bone disease from said individual; (b) screening said biological sample for one or more genetic indicators of a susceptibility for bone disease in said PTHrP gene of said individual; and (c) diagnosing said individual with respect to said susceptibility for bone disease based on the results obtained in step (b); wherein said individual is diagnosed with a risk for developing bone disease when one or more genetic indicators of a susceptibility for bone disease is detected.

In accordance with another aspect of the present invention, there is provided a method of treating a patient identified with bone disease or a susceptibility therefore, said patient having a PTHrP gene; said method comprising: (a) characterizing the PTHrP gene of said patient with respect to variations detected within a variable number tandem repeat (VNTR) region of said gene; and, (b) selectively treating the patient with a treatment regime corresponding to a genotypic profile of said patient; wherein said genotypic profile is characterized with respect to the detected variations within the VNTR region of said gene.

In accordance with still a further aspect of the present invention there is provided an isolated nucleic acid having a nucleotide sequence comprising one or more repeats of GTATATATA and/or ATATATATA wherein said nucleotide sequence has complimentarity to a region of a PTHrP gene. An isolated nucleic acid of the present invention may be used as a probe for a genetic indicator of bone disease. Alternatively, an isolated nucleic acid of the present invention may be employed as a therapeutic agent in connection with a treatment or prophylactic regime provided to target bone disease.

In accordance with yet another aspect of the present invention, there is provided a commercial package for use in providing a diagnosis of bone disease and/or a risk of developing bone disease in an individual, the commercial package comprising: (a) means for detecting at least one genetic indicator of bone disease and/or susceptibility for bone disease in a PTHrP gene of said individual; and (b) instructions for characterizing said at least one genetic indicator and correlating a diagnosis and/or prediction of bone disease.

As a further aspect, there is provided a method of diagnosing a male human for the susceptibility or predisposition to osteoporosis or osteopenia, the method comprising: a) amplification of a variable number tandem repeat (VNTR) region within an intronic region between exons VI and VII of a parathyroid hormone related peptide (PTHrP) gene in a biological sample from said human using primers corresponding to the sequences of SEQ ID NO: 6 and SEQ ID NO: 7 or complements thereof; b) measuring the length of the amplified VNTR region of said human; and c) diagnosing said human based on the results of step (b), wherein the presence of a VNTR region 252 base pairs in length is correlated with a diagnosis for susceptibility or predisposition to osteoporosis or osteopenia.

In embodiments, a bone disease of the present invention is a metabolic bone disease. More specifically, a bone disease of the present invention includes, but is not limited to osteoporosis, osteomalacia, osteopetrosis, Paget's disease, bone metastasis, and renal osteodystrophy.

In an embodiment, the characterization of a genetic indicator of the present invention comprises amplification of the VNTR region of a PTHrP gene, by PCR, for example, in accordance with methods well known in the art.

The invention further provides a use of a genetic indicator of the present invention to (a) diagnose bone disease in an individual; (b) determine if an individual has a predisposition to develop bone disease; or (c) both (a) and (b).

The invention further provides a method of treating an individual having PTHrP-gene, comprising screening the individual for a genetic indicator of bone disease; characterizing a genotypic profile of the individual based on identification of one or more genetic indicators in the PTHrP gene; and, treating the individual for bone disease if the genotypic profile of the individual is indicative of a risk of developing bone disease. According to an embodiment of the present invention, treating an individual may comprise developing a genotype-specific treatment regime for the individual. A genotypic profile of the present invention is preferably a PTHrP-specific genotypic profile. Accordingly, based on the genotypic profile of an individual, preferably the allelic polymorphism determined based on the allelic length of the VNTR region of the PTHrP gene, if an individual is identified to be at risk of developing osteoporosis, then said individual would benefit from an early BMD measurement to establish the baseline; and if confirmed to be osteopenic or osteoporotic, will benefit from prophylactic therapies ranging from supplemental Vitamin D and calcium intake to maintenance dose of an anti-resorptive agent such as alendronate (Fosamax) or a bone anabolic agent such as teriparatide (Forteo). Accordingly the present invention allows for the detection and characterization of an individual, having a PTHrP gene, wherein, depending on the allelic length of the VNTR region, as determined by the methods of the present invention, one skilled in the art would clearly appreciate that based on the characterization and results of the methods of the present invention, an individual would benefit from knowing his or her predisposition to bone disease, and as such would benefit from early preventive therapy with calcium, vitamin D or even other agents. For example, it has been observed in a preliminary clinical study that male osteoporotics have higher frequency of an allelic length of 252 base pairs, wherein the early diagnosis of said individuals would advantageously provide these osteoporotic, or osteoporosis-predisposed, individuals with the benefit of early diagnosis and prophylactic treatment or preventative therapy for bone disease. This data provides clear enabling data of the presence of the genetic indicator, i.e. in this case, the determined presence of a PTHrP VNTR length of 252 base pairs, in a patient that has bone disease, and more specifically, osteoporosis. It has also been observed that based on the method of diagnosis of the present invention, and the characterization of the allelic polymorphism determined, the treatment of the PTHrP+/− mice, i.e. animals predisposed to osteoporosis, with PTH (1-34) advantageously eliminates or reduces the manifestation of bone disease.

In accordance with another aspect of the present invention, there is provided a transgenic non-human mammal homozygous for a missing PTHrP gene (PTHrP^{-/-}) in osteoblast cells. In accordance with a preferred embodiment, the mammal is heterozygous for a missing copy of PTHrP gene (PTHrP^{+/-}) or homozygous for the absence of the PTHrP gene only in osteoblast cells, but not in non-osteoblast cells. Preferably, the transgenic non-human mammal of the present invention is suitable for studying the development, treatment and prophylaxis of bone disease. Such a transgenic non-human mammal may have further application in the screening of compounds and/or agents having therapeutic and/or prophylactic effect on bone disease(s).

The present invention also provides a transgenic non-human mammal heterozygous for a disrupted PTHrP gene (PTHrP+/−) in all cells of said mammal. In a preferred embodiment, the heterozygous mammal of the present invention lacks a copy of the PTHrP gene in all cells, i.e. in both osteoblast and non-osteoblast cells, wherein all cells of said mammal are heterozygous for disrupted PTHrP (i.e. PTHrP^{-/+}).

In accordance with another aspect of the present invention, there is provided a single-stranded nucleic acid having a nucleotide sequence comprising one or more repeats of GTATATATA or ATATATATA wherein said nucleotide sequence has complimentarily to a region of a PTHrP gene.

In accordance with another aspect of the present invention, there is provided a single-stranded oligonucleotide comprising of at least GTATATATA or ATATATATA for use in identifying candidate compounds having the ability to modulate PTHrP expression.

In accordance with another aspect of the present invention, there is provided a single-stranded oligonucleotide comprising two or more repeats having a sequence of GTATATATA or ATATATATA for use in identifying candidate compounds having the ability to modulate PTHrP expression.

In accordance with another aspect of the present invention, there is provided a single-stranded oligonucleotide comprising the sequence of SEQ ID NO: 1, or the complement thereof, for use in identifying candidate compounds having the ability to modulate PTHrP expression.

In accordance with another aspect of the present invention, there is provided an isolated nucleic acid comprising a sequence that hybridizes under stringent conditions to a hybridization probe the nucleotide sequence of which consists of SEQ ID NO: 1 or the complement of SEQ ID NO: 1.

In an embodiment, the somatic cells of the non-human mammal comprise osteoblast-specific PTHrP disruption means, such as a Cre-LoxP technology as described further herein below, capable of disrupting a PTHrP gene or a portion thereof specifically in osteoblast cells without disrupting the PTHrP gene in non-osteoblast cells. In an embodiment, such means comprise: (a) a Cre recombinase gene under control of an osteoblast-specific promoter (e.g. the type 1 collagen promoter); and (b) loxP sites which flank the PTHrP gene or a portion thereof (e.g. exon 4) (PTHrP flox/flox) (He et al. Endocrinology).

All references cited herein are incorporated herein by reference to the same extent as if each individual publication, patent application or issued patent was specifically and individually indicated to be incorporated by reference in its entirety for all purposes.

### BRIEF DESCRIPTION OF THE DRAWINGS

Further features and advantages of the present invention will become apparent from the following detailed description, taken in combination with the appended drawings, in which:

FIG. 1: Results of microCT analysis of bone from normal (left specimen) and heterozygous PTHrP (+/−) mice (right specimen), showing the diminished content of trabecular bone in the mutant animals.

FIG. 2: Measured bone volume over total volume (BV/TV) in normal and heterozygous PTHrP (+/−) mice. B.V. Bone Volume, Tb.N, number of bone trabecules; Tb.Th., thickness of bone trabecules.

FIG. 3: Results of analysis, in wild-type and heterozygous PTHrP (+/−) mice, of spacing between trabecules (Tb.Sp.), degree of anisotropy (DA), total volume (TV) and structure model index (SMI), features that describe the 3-D architecture of the bone.

FIG. 4: Analysis of bone from wild-type and heterozygous PTHrP (+/−) mice via calcein staining followed by tetracycline (10 days later) staining. The distance between the two lines (Panels C and D) can be used to calculate the rate of bone formation, which is markedly diminished in the mutant mice.

FIG. 5: Analysis of differentiation into osteoblasts. Bone marrow from wild-type (left) and heterozygous PTHrP (+/−) (right) animals was extracted, cultured and induced to differentiate into osteoblasts (bone-forming cells), and subsequently stained with for alkaline phosphatase activity, a marker of osteoblast differentiation.

FIG. 6: Schematic representation of preparation of mice in which PTHrP is specifically inactivated in osteoblasts, according to an embodiment of the invention.

FIG. 7: Analysis of bone from control mice (PTHrPflox/flox) and mutant mice lacking PTHrP in osteoblasts (PTHrP flo/flox/creColl).

FIG. 8: Analysis of osteoblasts from control mice (left panel) and mutant mice lacking PTHrP in osteoblasts (right panel) via staining of apoptotic nuclei.

FIG. 9: Structure of PTHrP genes from different species.

FIG. 10: PTHrP VNTR region (SEQ. ID NO: 5). Regions corresponding to oligonucleotide primers used for PCR amplification are underlined. Tandem repeats (G/ATATATATA) are shown in bold.

FIG. 11: Prevalence of PTHrP alleles in the general population. It is indicated on the figure that 16 of 19 osteoporotic male subjects tested (i.e. 84% of subjects) possessed the 252 bp allele.

FIG. 12: Analysis of the trabecular bone in wild type and PTHrP^{+/-} mice; three month old wild type (open bars) and PTHrP^{+/-} (solid bars) litter mates were injected subcutaneously with either vehicle or PTH 1-34 (40 μg/kg/day) 6 times per week for 12 weeks at which time PCT analysis of the skeleton was performed; results of the analysis in wild type and heterozygous PTHrP^{+/-} mice of measured BV/TV (bone volume over total volume); Tb.N (number of bone trabecules); Tb.Th. (thickness of bone trabecules); Tb.Sp. (spacing between trabecules); Connectivity; DA (degree of anisotropy); and SMI (structure model index).

FIG. 13: Agarose gel of amplified PTHrP VNTR separated according to molecular size on a 2.0% agarose gel, stained with ethidium bromide and visualized under UV light; Gene ruler 100 bp DNA ladder from Fermentas was used as a standard ladder; an aliquot of 3 μl of PCR product and 1 μl loading buffer was loaded onto the agarose gel.

### DETAILED DESCRIPTION OF THE PREFERRED EMBODIMENT

Osteoporosis is a very prevalent disorder with substantial morbidity and mortality affecting the aging population, as are many other bone diseases. A simple test that predicts early the potential risk for an individual to develop a bone disease, such as osteoporosis, for example is very much needed and would be greatly served in accordance with embodiments of the present invention.

As discussed further herein below, it has been identified in accordance with the present invention that the length of a variable number tandem repeat (VNTR) region in the PTHrP gene serves as a genetic indicator of bone disease, wherein the genetic indication of bone disease is based on allelic polymorphisms in the VNTR of the PTHrP gene, wherein various VNTR allelic lengths are used to diagnose the presence, predisposition or susceptibility to bone disease in an individual.

A study conducted in accordance with the present invention examines the predictive value of the allelic length of a VNTR region in the PTHrP gene on bone mineral density (BMD) in four groups of patients with normal and decreased BMDs: (1) osteoporotic males, and (2) osteoporotic premenopausal women, with both groups having a genetic etiology as the cause of osteoporosis, (3) healthy males, (4) healthy premenopausal women showing no signs of osteoporosis. The basis of this study proposes that allelic variations of the VNTR region of the PTHrP gene give rise to variable expression of the PTHrP protein within the bone microenvironment. Specifically, decreased expression of the PTHrP protein is herein correlated with decreased bone formation, and therefore diminished BMD, as discussed further herein below. Based on a characterization of an individual's genotypic profile, i.e. the allelic variation determined, with respect to bone disease, as provided in accordance with the identification of genetic indicators of the present invention, a corresponding genotype-specific treatment regime can be prescribed that is tailored to an individuals' genotype.

Intermittent administration of parathyroid hormone related peptide (PTHrP) in vivo has an anabolic effect on bone formation (Stewart et al. J. Bone Miner Res 15:1517, 2000). The precise molecular and cellular basis for the anabolic actions of PTHrP are unclear, and efforts to elucidate these mechanisms in PTHrP knockout mice have been compromised due to the perinatal lethality of these animals prior to the present invention. To overcome this obstacle, described herein is a novel system enabling the selective inactivation of PTHrP specifically in osteoblasts. Specifically, Cre-mediated recombination is used to selectively disrupt PTHrP expression in mouse osteoblasts. To generate mice with osteoblasts lacking the PTHrP gene (PTHrPflox/flox;crecol I), PTHrP^{+/flox} (exon 4 of PTHrP flanked by loxP sites) mice carrying the Cre recombinase transgene under the control of the 2.3-kb fragment of the murine pro-α1(I) collagen gene promoter (Col I) were crossed with mice homozygous for the floxed PTHrP allele. To genotype mice, flox and Cre recombinase gene were detected by Southern blotting, as previously described (He et al., 2001).

This has been substantiated by several modalities in accordance with the present invention including classic histomorphometry and bone densitometry (DEXA was used in patients) as well as by more advanced techniques, including specifically microcomputed tomography (mCT) of bone. The studies described herein utilize transgenic mice that have been generated in which the PTHrP gene has been selectively removed from osteoblasts, and not from other cells in the animal, i.e. the animals osteoblast cells are all PTHrP^{-/-}. These mice are shown to develop premature, severe osteoporosis. It is concluded in accordance with the present invention that the level of PTHrP expression within the bone microenvironment, specifically the osteoblasts, is critical for preventing osteoporosis. Furthermore, transgenic non-human mammals of the present invention further provide a novel system for studying bone disease that is very phenotypical of the actual human disease condition. Transgenic non-human mammals of the present invention also serve as novel transgenic non-human mammal models for studying the effects of candidate treatment regimes of candidate treatment regimes, such as compounds and/or agents for their effectiveness in treating, preventing or ameliorating the progression of bone disease, such as compounds and/or agents for their effectiveness in treating, preventing or ameliorating the progression of bone disease. In accordance with a preferred embodiment of the present invention, a system for evaluating and/or studying candidate therapeutics that modulate the expression of PTHrP in mammalian cells is provided. Osteoblast-specific PTHrP knock-out mammals of the present invention may be homozygous (PTHrP^{-/-}), where said homozygous mammals are missing both copies of the PTHrP gene specifically in osteoblast cells only, or heterozygous (PTHrP^{-/+}), where said heterozygous mammals are missing one copy of the PTHrP gene in all the cells of said mammals, i.e. missing a copy of the PTHrP gene in osteoblast and non-osteoblast cells.

The teachings and studies of the present invention describe and demonstrate that a specific region in the human PTHrP gene, herein referred to as a Variable Number of Tandem Repeats (VNTR) region, has a role in the regulation of PTHrP expression, and accordingly in bone disease. In accordance with the present invention, the VNTR region of the PTHrP gene is identified as a novel marker for bone disease as herein described. In particular, a genetic indicator of bone disease and/or susceptibility for bone disease of the present invention may comprise the VNTR region of the PTHrP gene or a portion thereof. That is to say, in accordance with the present invention, a genetic indicator of bone disease is the determined allelic length of the VNTR region of the PTHrP gene, wherein variations in allelic length allow for a means of diagnosing the presence, susceptibility or predisposition to bone disease in a subject.

While the existence of the VNTRs has been described, no role or function has been ascribed to it prior to the studies described herein. In the general population, this region varies in length from 252 to 460 nucleotides amongst individuals. The VNTR region may therefore serve as a regulatory region for PTHrP gene transcription, wherein transcription factors and/or other proteins may bind to this region and accordingly effect or modulate transcription and protein expression. This region may therefore serve as a regulatory region for PTHrP gene transcription, as transcription factors and or other proteins may bind to this region.

According to an embodiment of the present invention, novel oligonucleotides are herein provided. According to a preferred embodiment, an oligonucleotide of the present invention is an osteoblast-specific oligonucleotide. Preferably, an oligonucleotide of the present invention comprises at least a 9-mer nucleotide sequence as disclosed herein. More preferably, an oligonucleotide of the present invention includes a tandem repeat sequence comprising at least two 9-mer nucleotide sequences as disclosed herein. Oligonucleotides of the present invention are useful in characterizing novel therapeutic targets. An oligonucleotide of the present invention may be generated in accordance with well-known methodologies known in the art. Furthermore, an oligonucleotide of the present invention may be employed as a hybridization probe in accordance with embodiments of the present invention, and according to well known methods for achieving hybridization, such as those exemplified herein below.

In an analysis of a number of male subjects diagnosed with a genetic basis for osteoporosis, it was identified, in accordance with the present invention that 16 out of the 19 patients possessed a high risk allele, containing a shorter VNTR-containing region in the PTHrP gene, and likely less PTHrP expression within their skeletal microenvironment, consistent with the animal findings that lower PTHrP levels in osteoblasts is associated with the propensity to develop premature and severe forms of osteoporosis. Accordingly, in accordance with an embodiment of the present invention, a genetic indicator of bone disease and/or a predisposition for bone disease may be an allele of the PTHrP gene containing a VNTR region that is shorter than that of the average population Accordingly, in accordance with one embodiment of the present invention, a genetic indicator of bone disease and/or a predisposition for bone disease may be an allele of the PTHrP gene containing a VNTR region that is shorter than that of the average population. More preferably, a proportional relationship between VNTR length and predisposition to bone disease or severity of bone disease is identified in accordance with embodiments of the present invention. For example, the longer a VNTR region within a PTHrP gene of an individual of interest, the less likely that individual is to develop a bone disease; while a shorter VNTR region within the PTHrP gene, the more likely an individual is to develop the disease. Alternatively, a diagnosis on the severity of an individual's bone disease may be provided in accordance with an embodiment of the present invention, whereby when an individual identified as having bone disease or symptoms thereof is characterized as having a shorter allele of the VNTR region of the PTHrP gene, the individual may be diagnosed with a more severe form of bone disease than an individual identified as having a longer allele. Furthermore, an individual may be selectively treated for bone disease or a predisposition for bone disease in accordance with a genotypic characterization of the present invention. Preferably, a personalized or specific treatment regime corresponding with the individual's genotype is determined and prescribed.

A genetic test specific for characterizing alleles of the PTHrP gene, to identify individuals at risk for developing bone disease and in particular a genetic test to identify a predisposition for osteoporosis has not been described prior to the studies described herein. Accordingly, the present invention provides a method of identifying the risk of developing osteopenia or osteoporosis based on genetic indicators.

Specifically, described herein is a correlation between certain alleles of the PTHrP gene, also herein referred to as genetic indicators, or allelic polymorphisms based on the allelic length of the PTHrP gene, and bone disease. In accordance with the present invention, alleles of the PTHrP gene have been identified as genetic indicators of bone disease and/or genetic indicators of a predisposition for bone disease. The alleles of the present invention are defined in part based on the size of a genomic region of the PTHrP gene, herein referred to as a VNTR-containing region, and further in part based on the number of VNTRs contained within this region. A high risk allele, which is indicative of a greater risk of metabolic bone disease, comprises a shorter VNTR-containing region containing fewer VNTRs. In embodiments, the bone disease is more preferably a metabolic bone disease. According to a preferred embodiment of the present invention a metabolic bone disease may be selected from the group consisting of osteoporosis, osteomalacia, osteopetrosis, Paget's disease, and renal osteodystrophy.

The sequence of each VNTR may comprise a sequence of RTATATATA, (SEQ ID NO: 2), wherein, ‘R’ indicates that the first position of each VNTR may be a G or A. That is to say, a VNTR sequence may comprise the sequence GTATATATA or ATATATATA.

Accordingly, nucleic acid sequences and/or oligonucleotides comprising one or more repeats of the nucleotide sequences outlined above are provided in accordance with the present invention. Isolated nucleic acids of the nucleotide sequences outlined above are provided in accordance with the present invention. Isolated nucleic acids and/or oligonucleotides of the present invention may be employed as detection means, for example, as hybridization probes, for identifying genetic indicators of bone disease. Furthermore, the probes of the present invention are useful in identifying compounds with therapeutic or prophylactic potential in treating bone disease or a predisposition thereto. Alternatively, nucleic acid sequences and/or oligonucleotides of the present invention may be administered in vivo as a preferred therapeutic and/or prophylactic treatment regime for combating bone disease. The nucleic acid sequences and oligonucleotides of the present invention are prepared or isolated in accordance with methodologies well known in the art, for example, some of such methodologies are described in the teachings of Sambrook et al. (1989, Molecular Cloning—A Laboratory Manual, Cold Spring Harbour Laboratories); Ausubel et al. (1994, Current Protocols in Molecular Biology, Wiley, New York); and Glick et al. (1994 Molecular Biotechnology—Principles and Applications of Recombinant DNA, ASM, Washington, D.C.). When employed as hybridization probes, the nucleic acid sequences and oligonucleotides of the present invention preferably hybridize with nucleic acids under stringent hybridization conditions, as well known in the art or as exemplified hereinbelow.

A correlation between the alleles of the VNTR region of the PTHrP gene with the level of PTHrP expression have been identified in accordance with the present invention. For example, the present invention has shown that alleles comprising longer VNTR-containing regions within the PTHrP gene correlate with higher levels of PTHrP. Furthermore, an increase in the number of repeats within the VNTR-region of the PTHrP gene have been correlated with higher levels of PTHrP expression, and thus, better bone formation. Accordingly, one aspect the invention provides for a genotyping assay for identifying a genetic indicator (i.e. a high risk allele, namely a high risk allelic length) that is associated with low levels of PTHrP expression, to provide an indication that an individual, having the genetic indicator, is at risk for developing bone disease. In addition, the invention provides assays for identifying a genetic indicator associated with high levels of PTHrP (i.e. a low risk allele) to provide an indication that an individual having such an indicator is less likely to develop bone disease. For example, a 252 bp allele, that is to say an allelic length of 252 base pairs of the VNTR containing region of a PTHrP gene is shown in accordance with the present invention to have a positive correlation with bone disease, i.e. the presence of osteoporosis or the predisposition thereto in individuals having an allelic length of 252 base pairs for the VNTR region of the PTHrP gene.

In accordance with this aspect of the present invention, a genetic indicator is characterized on the basis of length to have a genotypic basis for contributing to a disease or non-disease state in an individual whose genetic composition is determined to contain that indicator.

According to one embodiment, a degree of severity of a disease state may be characterized in accordance with a genetic indicator of the present invention.

The invention may be utilized to diagnose and/or treat individuals identified as being at risk of developing bone disease. For example, such individuals may exhibit one or more symptoms of bone disease or related conditions, or possess one or more of a group of risk factors associated with bone disease and/or related conditions. Individuals may exhibit one or more symptoms of bone disease or related conditions, or possess one or more of a group of risk factors associated with bone disease and/or related conditions. Individuals may for example be identified as at risk of bone disease on the basis of epidemiological criteria such as sex, age, socioeconomic factors or family history, on the basis of which an assessment may be made that the individual of interest is more likely than other persons to suffer from bone disease. Physicians typically diagnose bone disease based on the overall pattern of symptoms, medical history, family history, medications, physical exam, imaging methods (measurement of BMD, Bone Mineral Density) and a variety of blood and urine tests for determining bone integrity.

Thus, the present invention provides methods for the diagnosis and prediction of metabolic bone disease, via assessing a PTHrP gene for predetermined genetic indicators appearing therein (the nature of a PTHrP allele present in a subject). Upon identification of a genetic indicator of the present invention, an individual's genotypic profile pertaining to bone disease can be characterized. A subject of the present invention is preferably a mammal, and more preferably a human. A subject of the present invention may be a subject at risk for metabolic bone disease.

In an embodiment, a genetic indicator is characterized based on the nature of the PTHrP VNTR containing region present in the allele of the PTHrP gene.

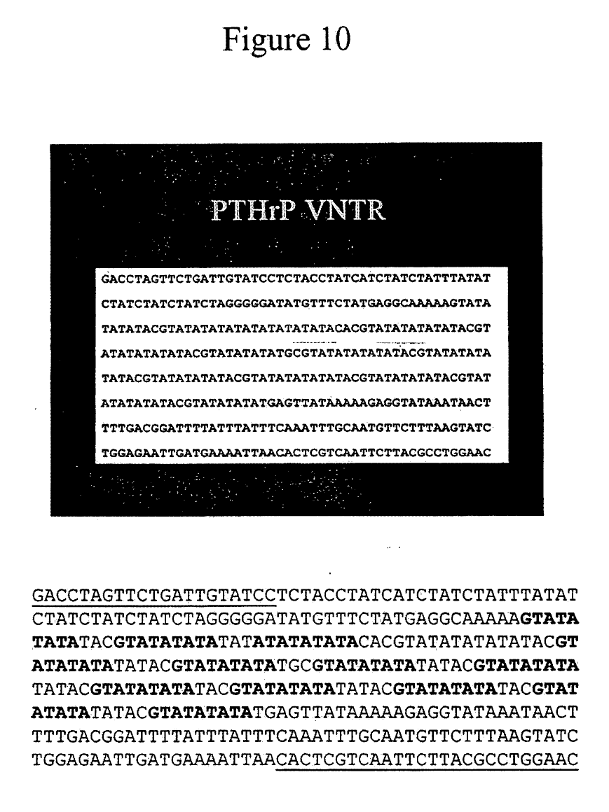

Accordingly, the invention provides a method of diagnosis or prediction of bone disease, the method comprising: a) obtaining a biological sample from said individual, said sample suitable for detecting a parathyroid hormone related peptide (PTHrP) gene of said patient therein; b) screening for one or more genetic indicators of bone disease in said PTHrP gene of said individual; and c) diagnosing said individual with respect to risk for bone disease based on the results obtained in step (b). Thus, characterization of a genetic indicator identified in accordance with the present invention shall allow diagnosis of a bone disease or indicate that the patient has a predisposition for bone disease. According to a preferred embodiment of the present invention, a genetic indicator of the present invention is characterized on the basis of length. More preferably, a genetic indicator of the present invention includes a VNTR containing region of the PTHrP gene. Variants of the VNTR containing region of the PTHrP gene are also herein referred to as alleles or allelic variants, in accordance with embodiments of the present invention. In an embodiment, the genetic indicator occurs within a variable number of tandem repeat (VNTR)-region within an intron of the PTHrP gene. In an embodiment the intron is between exons VI and VII of the PTHrP gene. The characterization of a genetic indicator, in an embodiment of the present invention; may be based on the number of VNTRs present in an allele of PTHrP gene (e.g. three 9-mer nucleotide sequence repeats). The characterization of a genetic indicator may, in a further embodiment of the present invention, be based on the identification of an allele selected from the group consisting of a 252 bp, 288 bp, 332 bp, 356 bp, 378 bp, 393 bp, 414 bp and 460 bp allele comprising a variable number of tandem repeat (VNTR)-region of the PTHrP gene. A PTHrP allele (or a genetic indicator based thereon) may be identified by various methods known in the art. For example, an allele comprising a VNTR region may be identified by amplifying this region via polymerase chain reaction (PCR) to obtain a PCR product, and examining the length of the PCR product obtained. Suitable oligonucleotide primers may be used for such amplification, for example, those shown in FIG. 10, as obtained from SHELDON BIOTECH, Montreal, PQ.

An allele may be identified, for example, by direct sequencing of the PTHrP gene or a region thereof, such as the VNTR containing region. Another method for identifying the PTHrP may be restriction fragment length polymorphism analysis (RFLP), wherein, for example, by amplifying a nucleic acid sequence comprising the PTHrP gene or a region thereof, such as a VNTR containing region, by polymerase chain reaction (PCR) to obtain a PCR product, digesting the PCR product with a restriction enzyme to obtain a restriction digest product; and examining the length of the restriction digest product(s) produced, such as by gel electrophoresis, for example. Other methods may utilize hybridization of allele-specific probes under hybridization conditions which are optimized so that allelic differences may be detected. Microarray based methods may also be used, such as for example as described in U.S. Pat. Nos. 5,858,659 (Sapolsky et al.; Jan. 12, 1999) or 6,223,127 (Berno; Apr. 24, 2001).

In embodiments, a PTHrP allele is identified in a biological sample obtained from a subject, such as a tissue or body fluid of said subject. Suitable tissue or body fluids include but are not limited to blood, plasma, lymphocytes, epithelial cells, osteoblasts, bone marrow stromal cells, and fibroblasts. A biological sample of the present invention comprises DNA. Preferably, an individual's DNA can be detected and/or amplified from a biological sample of the present invention. DNA amplification and detection are carried out in accordance with methodologies well-known in the art, some of which are exemplified in the references herein incorporated by reference.

In an embodiment, a nucleic acid comprising a PTHrP gene or portion thereof is purified from the sample prior to identifying the PTHrP allele therein. In an embodiment, the purified nucleic acid is amplified prior to determining the PTHrP allele present. In an embodiment, the purified nucleic acid is amplified with primers which amplify a PTHrP gene or a fragment thereof. In certain embodiments, such amplification is performed via PCR using primers which are designed such that they hybridize to sequences on either side of (i.e. 5′ to and 3′ to) the VNTR-containing region, for example, the primers shown in FIG. 10.

The invention further relates to a use of an allele of a PTHrP gene, as described above, for the diagnosis and/or prediction of bone disease. In accordance with a preferred embodiment, the present invention provides a hybridization probe having sequence homology to at least a 9-mer nucleotide sequence of an allele of a PTHrP gene.

The invention further relates to commercial packages or kits for carrying out the diagnostic and predictive methods noted above, comprising the appropriate above-mentioned reagents (i.e. primers or probes, for example) together with instructions for methods for using such a commercial package for the purpose of diagnosing and/or predicting susceptibility for bone disease.

Accordingly, the invention further provides a commercial package or kit for use in the diagnosis and/or predicting susceptibility for bone disease. The commercial package or kit of the present invention may comprise means for detecting a genetic indictor of bone disease in a PTHrP gene of a subject of interest, together with instructions for characterizing said genetic indictor as a positive or negative indicator of bone disease and/or susceptibility thereto. More preferably, the package or kit of the present invention includes quantitative correlation information relating predetermined genetic indicators to levels of disease severity and/or degrees of susceptibility thereto. In accordance with a preferred embodiment, genotyping is performed on amplified PTHrP VNTR gene isolated from a test sample, preferably a patient's blood sample, wherein the presence or absence of particular alleles is determined. The detection is preferably performed using standard ladders of oligos varying in size, for example, from 100 bp to 1,000 bp. In accordance with a preferred exemplified characterization of a sample in accordance with the present invention, genotyping involves collection of a sample, preferably blood samples from human subjects, isolation of buffy coat by centrifugation of the blood, DNA extraction using the commercially available blood DNA mini kits, PCR amplification, agarose gel electrophoresis and genotyping of individual bands against standard ladders to determine the alleles, or genetic indicators or genetic markers present in the sample. For example, if the amplified PCR fragment has a determined length of, for example, 252 base pairs, this would be a genetic indicator of the presence of bone disease or a genetic indicator of the susceptibility for bone disease. A kit or commercial package of the present invention would comprise instructions relating to the use the kit for genotyping a test sample, and instructions relating to assessing the risk of bone disease, preferably osteoporosis, depending on the presence or absence of particular alleles, for example the presence of a 252 bp VNTR length.

In another aspect, the invention provides a method of treating a patient identified with bone disease or a susceptibility therefore, said patient having the PTHrP gene; said method comprising: (a) characterizing the PTHrP gene of said patient with respect to variations detected within a variable number tandem repeat (VNTR) region of the PTHrP gene; and, (b) selectively treating the patient with a customized treatment regime corresponding to the detected variations within the VNTR region of said gene. In accordance with preferred embodiment, based on the genotypic profile of an individual, as determined by the characterization of the PTHrP gene, and more preferably on the VNTR genetic indicators, or allelic lengths determined, if the patient's test sample is identified to comprise genetic indicators relating to having a risk of developing osteoporosis, then said individual would benefit from an early BMD measurement to establish the baseline, and if confirmed to be osteopenic or osteoporotic, the patient may benefit from prophylactic therapies ranging from supplemental Vitamin D and calcium intake to maintenance dose of an anti-resorptive agent such as Fosamax or a bone anabolic agent such as Forteo.

A variation in the PTHrP gene may be in the length of the VNTR region within the intron between exons VI and VII. Thus, such a variation would serve as a suitable genetic indicator in accordance with the present invention. The presence of a high risk allele (or genetic indicator) of the present invention may for example be taken as indicative of susceptibility or a predisposition to bone disease or to a more severe form of bone disease. More preferably, the present invention provides a method for selectively treating bone disease and/or a predisposition thereto according to an individual's genotype. That is, a genotype specific treatment or prophylactic regime to combat bone disease may be provided in accordance with the present invention. According to this embodiment of the present invention, a characterization of an individual's genotype is made according to genetic indicators of the present invention and a corresponding treatment or prophylactic regime is devised based thereon. For example, an individual identified to have a 252 bp allele of the PTHrP gene, will be prescribed a corresponding treatment regime that is different from that prescribed to another individual identified to have 460 bp allele of the PTHrP gene, according to an embodiment of the present invention.

In accordance with various aspects of the invention, a patient may be treated for bone disease. For example, treating a patient for bone disease may comprise administering to the patient an effective amount of a compound or medicament. An effective amount of a medicament may be a therapeutically effective amount or a prophylactically effective amount. A “therapeutically effective amount” refers to an amount effective, at dosages and for periods of time necessary, to achieve the desired therapeutic result, such as reducing signs and symptoms of bone disease and/or delaying structural damage of bone. A therapeutically effective amount of a therapeutic may vary according to factors such as the disease state, age, sex, and weight of the individual, and the ability of the therapeutic to elicit a desired response in the individual. Dosage regimens may be adjusted to provide the optimum therapeutic response. A therapeutically effective amount is also typically one in which any toxic or detrimental effects of the therapeutic are outweighed by the therapeutically beneficial effects. A “prophylactically effective amount” refers to an amount effective, at dosages and for periods of time necessary, to achieve the desired prophylactic result, such as reducing signs and symptoms of bone disease and delaying structural damage of bone. A prophylactic dose may be used in subjects prior to or at an earlier stage of disease, and a prophylactically effective amount may be more or less than a therapeutically effective amount in some cases.

Medicaments for treating bone disease may, for example, include drugs approved by the FDA for treating patients with varying degrees of bone disease, such as drugs that reduce signs and symptoms of bone disease and delay structural damage of bone disease in patients. Such drugs may for example include: estrogen, alendronate, residronate, calcitonin, and parathyroid hormone.

In one aspect, the invention relates to a use in gene therapy of an PTHrP nucleic acid. The PTHrP nucleic acid may be delivered by a therapeutically acceptable gene therapy vector to modify a patient's PTHrP allelic profile. Gene therapy may for example be used to replace a high risk PTHrP allele with a low risk PTHrP allele or enhance the expression of the PTHrP protein.

Gene therapy vectors may for example be an adeno-associated vector (AAV). Such a vector may comprise for example: a 5′ inverted terminal repeat (ITR); a promoter, such as a CMV enhancer-promoter with a osteoblast-specific enhancer; an intron; a 3′-untranslated region (3′-UTR); a polyadenylation signal, such as an SV40 polyadenylation signal; and a 3′-ITR. For gene therapy vectors, the dosage to be administered may depend to a large extent on the condition and size of the subject being treated as well as the therapeutic formulation, frequency of treatment and the route of administration. Regimens for continuing therapy, including dose, formulation, and frequency may be guided by the initial response and clinical judgment. The parenteral route of injection into the interstitial space of tissue may be preferred, although other parenteral routes, such as inhalation of an aerosol formulation, may be required in specific administration. In some protocols, a formulation comprising the gene and gene delivery system in an aqueous carrier is injected 30 into tissue in appropriate amounts. The tissue target may be specific, for example the muscle or liver tissue, or it may be a combination of several tissues, for example the muscle and liver tissues. Exemplary tissue targets may include liver, skeletal muscle, heart muscle, adipose deposits, kidney, lung, vascular endothelium, epithelial and/or hematopoietic cells and bone cells. A nucleic acid of the invention may be delivered to cells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., 15 from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson et al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel et al. (1991) Proc. Natl. Acad. Sci. USA 88:8850; Cristiano et al. (1993) Proc. Natl. Acad. Sci. USA 90:2122-2126). Defective retroviruses are well characterized for use as gene therapy vectors (for a review see Miller, A. D. (1990) Blood 76:271). 30 Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:76407644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

For use as a gene therapy vector, the genome of an adenovirus may be manipulated so that it includes a PTHrP nucleic acid, but is inactivated in terms of its ability to replicate in a normal lytic viral life cycle. See for example Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are well known to those skilled in the art. Recombinant adenoviruses are advantageous in that they do not require dividing cells to be effective gene delivery vehicles and can be used to infect a wide variety of cell types, including airway epithelium (Rosenfeld et al. (1992) cited supra), endothelial cells (Lemarchand et al. (1992) Proc. Natl. Acad. Sci. USA 89:6482-6486), hepatocytes (Herz and Gerard (1993) Proc. Natl. Acad. Sci. USA 90:2812-2816) and muscle cells (Quantin et al. (1992) Proc. Natl. Acad. Sci. USA 89:2581-2584).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et. al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in 30 the invention.

General methods for gene therapy are known in the art, for example, U.S. Pat. No. 5,399,346 by Anderson et al. (incorporated herein by reference). A biocompatible capsule delivering genetic material is described in PCT Publication 95/05452 by Baetge et al. Methods of gene transfer into hematopoietic cells have also previously been reported (see Clapp, D. W., et al., Blood 78: 1132-1139 (1991); Anderson, Science 288:627-9 (2000); and, Cavazzana-Calvo et al., 288:669-72 (2000), all of which are incorporated herein reference).

The invention further relates to a transgenic non-human mammal to study bone disease and the mechanisms thereof. More particularly, a non-human transgenic mammal of the present invention may be useful in studying the role of a PTHrP in bone formation, the non-human mammal having a disrupted/inactivated PTHrP gene (or portion thereof) either specifically in osteoblast cells, as would be the case for PTHrPflox/flox;crecol I mammals (PTHrP^{-/-} only osteoblast cells) wherein said mammals lack both copies of the PTHrP gene lacking from all osteoblast cells, while present in non-osteoblast cells) or in all cells of the mammal, as would be the case for heterozygous PTHrP knock-out mammals (PTHrP^{-/+} in all cells) wherein said heterozygous mammals lack one copy of the PTHrP gene from all cells, i.e. for both osteoblast and non-osteoblast cells), or heterozygous PTHrP knock-out mammals (PTHrP^{-/+} only in osteoblast cells) wherein said heterozygous mammals lack one copy of the PTHrP gene in osteoblast cells).

Accordingly, the present invention provides a number of transgenic mammals that may be used to study bone disease or may be used to screen compounds that may be potentially therapeutic compounds for the treatment of bone disease. As noted, said transgenic mammals may be: (a) PTHrP^{-/-} homozygous mammals in only osteoblast cells (PTHrPflox/flox;crecol I); (b) PTHrP^{-/+} heterozygous mammals in all cells; (c) PTHrP^{-/+} heterozygous mammals in only osteoblast cells. In preferred embodiments, the PTHrP^{-/-} homozygous mammals in only osteoblast cells (a) and the PTHrP^{-/+} heterozygous mammals in all cells (b) are used to study bone disease, and to screen for therapeutic compounds in the treatment of bone disease.

Furthermore, a non-human transgenic mammal of the present invention may be employed to screen compounds and/or agents for their effectiveness in modulating PTHrP expression and/or in treating, preventing or slowing the progression of bone disease. When patients requiring bone formation or repair are treated with a compound shown to increase PTHrP expression, PTHrP production by osteoblasts will be stimulated, resulting in bone formation and repair. Therefore, compounds identified in accordance with this embodiment of the present invention to modulate PTHrP expression may be used in the treatment of bone disease, bone breakdown, bone trauma, underdevelopment of bone and other conditions where PTHrP production is desired.

In an embodiment, somatic cells of a non-human mammal comprise PTHrP gene disruption means, for example, means capable of deleting/splicing out or inactivating a PTHrP gene, a portion thereof, or a nucleic acid sequence substantially identical in thereto, specifically in osteoblast cells. “Specifically in osteoblast cells” as used herein refers to removal or inactivation in osteoblast cells with substantially no removal or inactivation in non-osteoblast cells. In an embodiment, the mammal is a rodent, in a further embodiment, a mouse.

In an embodiment, the above-noted PTHrP gene disruption means comprises (a) a Cre recombinase gene osteoblast specific transcriptional regulatory region, such as a promoter; and (b) loxP sites which flank the PTHrP gene or a portion thereof. In an embodiment, the osteoblast-specific promoter is the type 1 collagen promoter.

As described in the Examples below, such animals may be used to study a number of PTHrP-dependent phenotypes in bone tissue, as well as having applications as described above. For example, such animals may be useful for the study of bone disease, bone formation, bone breakdown and bone trauma and for the discovery and development of therapeutics for bone disease. In particular, the transgenic non-human mammals of the present invention have particular application in the screening of potential therapeutic compounds and/or agents for effectiveness in treating, preventing or ameliorating bone disease In particular, the transgenic non-human mammals of the present invention have particular application in the screening of potential therapeutic compounds and/or agents for effectiveness in treating, preventing or ameliorating bone disease and/or bone conditions. A transgenic non-human mammal of the invention is advantageous in this regard as it provides a viable animal amenable to study over longer experimental periods, and still provides a homozygous null PTHrP −/− background in osteoblast cells, (or heterozygous PTHrP −/+ in all cells) allowing the study of bone development and disease with respect to PTHrP function. For example, PTHrP flox/flox/crecolI mice will be treated with the experimental agents or vehicle alone for a period of one to two months and then sacrificed to have their skeletons examined by various techniques including BMD, histology, histomorphometry, immunohistochemistry etc. Increased bone density and bone micro-architecture of experimental versus vehicle-treated animals will be considered significant.

Accordingly, the present invention provides a method of diagnosing bone disease in an individual, method comprising (a) obtaining a biological sample comprising DNA (b) screening sample for genetic indicators in PTHrP gene and (c) diagnosing individual based on result of (b). Preferably, screening involves looking for genetic indicator; wherein said genetic indicators comprise the VNTR region of the PTHrP gene; the screen may additionally use probes specific to the said genetic indicators.

The present invention also provides the use of an allele, allelic variations, of PTHrP gene as an indicator of bone disease/disposition; wherein the genetic indicator relates to the VNTR region of PTHrP gene; wherein, preferably, the VNTR region has variable number of tandom 9-mer oligonucleotide repeats; wherein the 9-mer repeats are selected from GTATATATA and/or ATATATATA. Depending on the allelic variations in the VNTR region, the VNTR region length may be 252 bp, 288 bp, 332 bp, 356 bp, 378 bp, 393 bp, 414 bp or 460 bp, wherein each VNTR variant length corresponds to an allelic variant determinant of the genotype of the individual.

The present invention also provides a method of diagnosing susceptibility for bone disease in an individual having a PTHrP gene, method comprising (a) obtaining a biological sample with genetic indicators predictive of bone disease (b) screening sample for such indicators (c) diagnosing individual based on result of (b). Preferably, genetic indicators comprise the VNTR region within an intron of PTHrP gene. Preferably, the characterization of VNTR region is based on length; wherein the length of the VNTR region correlates to susceptibility to bone disease. The VNTR region length may vary from 252 bp-460 bp, and is more specifically of a length of 252 bp, 288 bp, 332 bp, 356 bp, 378 bp, 393 bp, 414 bp or 460 bp. The VNTR region length varies based on the variable number of tandem repeats (9-mer oligonucleotides) comprising the region, wherein the 9-mer repeats are selected from GTATATATA and ATATATATA. Preferably, screening the biological sample for indicators comprises using probes specific to genetic indicators.

The present invention also provides a method of treating bone disease or bone disease susceptible patients or individuals having a PTHrP gene, the method of treating comprising (a) characterizing the PTHrP gene relative to the number of tandem repeats in VNTR of the PTHrP gene (b) treating the patient or individual with a treatment regime corresponding to the allelic determination based on the determined VNTR variations. The treatment regime of the present invention is preferably genotype-specific; treatment regime is variable over time; and treatment regime modulates expression of PTHrP gene to effect osteoblast activity. Preferably, the treatment regime enhances the VNTR region of PTHrP gene, wherein enhancing the VNTR region comprises the delivery of a variable number of copies of tandem repeats in vivo, where the tandem repeats of the VNTR region may be 9-mer tandem repeats having a sequence of GTATATATA or ATATATATA; wherein preferably the VNTR region is within an intron of PTHrP.

The present invention additionally provides a commercial package or kit for diagnosing/predicting bone disease in an individual, the package or kit comprising (a) means of detecting genetic indicators in PTHrP gene, wherein the detecting means detect variations in the VNTR region (b) instructions for characterizing genetic indicator and correlating with diagnosis/prediction of disease. The package or kit may additionally comprise means for determining length of genetic indicators. Moreover, package or kit may also comprise means for amplifying VNTR region of PTHrP. The detecting means additionally comprises probes specific to the VNTR region of the PTHrP gene; wherein the probes are specific to 9-mer tandem repeats having sequences GTATATATA or ATATATATA.

The present invention provides a transgenic non-human mammal homozygous for disrupted PTHrP (PTHrP^{-/-}) in osteoblast cells, wherein the homozygous knock-out mouse is lacking both PTHrP gene copies in osteoblast cells, and not in all cells of mammal. The somatic cells of the PTHrP^{-/-} homozygous (in osteoblast cell) mammal preferably comprise osteoblast-specific PTHrP disruption means, wherein the osteoblast-specific PTHrP disruption means are (a) Cre-recombinant gene controlled by osteoblast-specific promoter and (b) loxP sites flanking PTHrP gene or a portion. Preferably the osteoblast-specific promoter is type I collagen promoter. And additionally, the distrusted PTHrP gene portion is exon 4 of PTHrP gene. In accordance with a preferred embodiment of the present invention, the mammal is a mouse.

The present invention provides a transgenic non-human mammal heterozygous for disrupted PTHrP (PTHrP^{-/+}) in all cells of the mammal, wherein the heterozygous knock-out mouse is lacking one copy of the PTHrP gene in all cells, i.e. osteoblast and non-osteoblast cells, of the transgenic mammal. The somatic cells of the PTHrP^{-/+} heterozygous (in all cells) mammal preferably comprise osteoblast-specific PTHrP disruption means, wherein the osteoblast-specific PTHrP disruption means are (a) Cre-recombinant gene controlled by osteoblast-specific promoter and (b) loxP sites flanking PTHrP gene or a portion. Preferably the osteoblast-specific promoter is type I collagen promoter. And additionally, the disrupted PTHrP gene portion is exon 4 of PTHrP gene. In accordance with a preferred embodiment of the present invention, the mammal is a mouse.

The present invention additionally comprises PTHrP transgenic mammal(s) for use in the study of bone development and disease. The transgenic mammal(s) of the present invention may preferably be used to screen compounds/agents for the presence of a therapeutic/prophylactic ability for the potential use in the prevention/treatment/delaying of bone disease.

The present invention also provides a single-strand nucleic acid with nucleotide sequence of one of more 9-mer tandem repeats of GTATATATA or ATATATATA. The present invention also provides the use of this single-strand nucleic acid as a genetic indicator of bone disease or predisposition therefor. More preferably, the nucleic acid sequences has a length of 252 bp, 288 bp, 332 bp, 356 bp, 378 bp, 393 bp, 414 bp or 460 bp.

There is also provided a single-stranded oligonucleotide with sequence of GTATATATA or ATATATATA for use in identifying candidate compounds having the ability to modulate PTHrP expression.

The present invention also provides single-stranded oligonucleotide with two or more tandem repeats of sequences GTATATATA or ATATATATA for use in identifying candidate compounds having the ability to modulate PTHrP expression.

In a preferred embodiment, the single-stranded oligonucleotide having sequence SEQ ID NO: 1, or a compliment thereof, for use in identifying candidate compounds having the ability to modulate PTHrP expression and hence bone formation.

There is also provided an isolated nucleic acid having a sequence of SEQ ID NO: 1 or a compliment thereof, that hybridizes under stringent conditions to hybridize a hybridization probe.

In a preferred embodiment, the single stranded nucleotide sequences or oligonucleotides having at least one 9-mer tandem repeat having a sequence of GTATATATA and/or ATATATATA is used as a genetic indicator in the determination of VNTR allelic length variations, and for use in the identification of candidate compounds to modulate PTHrP expression.

Accordingly, the present invention provides methods of diagnosing the presence or susceptibility of bone disease, through the determination of variations in the length of the VNTR region of the PTHrP gene, as determined by the number of 9-mer tandem repeats present, the 9-mer tandem repeats having sequence GTATATATA or ATATATATA, wherein different VNTR region lengths define different allelic variants, preferably allelic variant defined by variant region length, more specifically lengths of 252 bp, 288 bp, 332 bp, 356 bp, 378 bp, 393 bp, 414 bp or 460 bp, wherein the determined length defines the specific allelic variant, wherein the determined length is as detected by specific probes, wherein the allelic length determined correlates to the presence or susceptibility of bone disease in a patient.

Accordingly, the present invention provides for the use of specific VNTR region lengths (252 bp, 288 bp, 332 bp, 356 bp, 378 bp, 393 bp, 414 bp or 460 bp) as allelic determinants which diagnose or detect the disposition to bone disease.

The provided commercial kit of the present invention is used for completing the methods described in the present invention, wherein the kit allows for the detection of variations in VNTR length, the amplification of the VNTR region, and the detection of the 9-mer tandem repeats in the VNTR using specific probes.

Furthermore, the present invention provides a homozygous PTHrP^{-/-} knock-out mammal (PTHrPflox/flox;crecol I), wherein only osteoblast cells are homozygous for disrupted PTHrP. The provided homozygous mammal is preferably used to study bone disease and to screen compounds that may be used for the treatment, prophylaxis, or delaying of bone disease.

Moreover, the present invention also provides a heterozygous PTHrP^{+/-} knock-out mammal, wherein all cells are heterozygous for disrupted PTHrP. The provided heterozygous mammal is preferably used to study bone disease and to screen compounds that may be used for the treatment, prophylaxis, or delaying of bone disease.

The use of PTHrP heterozygote (PTHrP^{+/-}) mice for studying and screening compounds and/or agents having therapeutic potential in the prevention and/or treatment of human diseases has been successfully shown. More specifically, the studies described below provide experimental supporting evidence that mice heterozygous for PTHrP gene inactivation (PTHrP^{+/-}) are an extremely valuable model of the genetic form of human osteoporosis and are invaluable products or tools for studying and screening compounds for not only studying the effects of said compounds on bone formation, but are also valuable models and tools that may preferably be used to screen compounds for the purpose of detecting compounds having therapeutic, prophylactic effects or may be used to delay bone disease. To confirm that the transgenic mammal of the present invention are in fact valuable study and screening models, the effects of confirmed bone anabolic agents, such as PTH (1-34), PTH (1-84), PTHrP (1-36), PTHrP (1-139), their cyclic and non-cyclic analogs, their peptidomimetic analogs, their small molecule drug analogs and other bone anabolic agents, PTH (1-34) was tested on the transgenic mammal(s) of the present invention, so as to confirm that the transgenic mammals provided in the present invention do in fact provide a true model of the genetic form of human osteoporosis.

The study of the present invention examined PTH and PTHrP interaction at the level of the osteoblast. The observation from the PTH^{-/-}/PTHrP^{+/-} double mutant mice suggest that while circulating PTH serves to maintain calcium homeostasis by resorbing bone, locally produced PTHrP acts to promote bone formation. The osteoporotic phenotype arising in PTHrP^{+/-} mice and PTHrPflox/flox cre col I mice has further confirmed this observation.

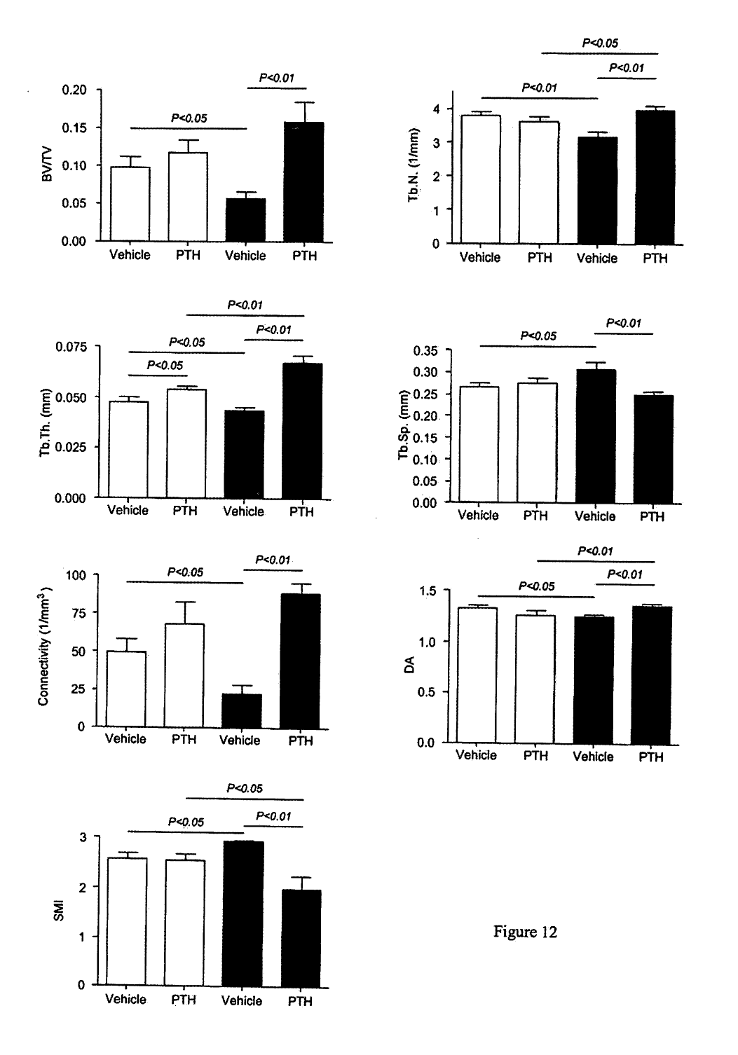

The study described aimed to examine how the anabolic action of daily administered exogenous PTH 1-34 is influenced in the setting of PTHrP haploinsufficiency (PTHrP^{+/-}). Three month old PTHrP^{+/-} male mice and wild type male litter mates were injected subcutaneously with either vehicle or PTH 1-34 (40 μg/kg/day) 6 times per week for 12 weeks at which time micro CT analysis of the skeleton was performed (as shown in FIG. 12). As illustrated, in wild type bones, administered PTH 1-34 had modest positive effects on all parameters examined, whereas profound anabolic changes were observed in treated bone specimens from PTHrP^{+/-} mice. BV/TV, a parameter of trabecular bone content, increased to 278% of baseline compared to 19% in wild type mice. Similarly, trabecular number (24% vs −4%), thickness (55% vs 12%), connectivity (306% vs 37%) and degree of anisotropy (9% vs −6%) were increased while trabecular spacing (−22% vs 3%) and structure model index (−32% vs −1%) were decreased in specimens from PTHrP haploinsufficient mice.

The observation of the present study, insight is provided as to how locally produced PTHrP and circulating PTH interact at the level of the osteoblast. For example, from the generation of double PTH^{-/-}/PTHrP^{+/-} mutant mice, it is apparent that the main action of PTH is to resorb bone and maintain calcium homeostasis while PTHrP promotes osteoblast activity/survival and thereby bone formation. Hence, a consequence of PTHrP haploinsufficiency at the level of the bone is the profound osteoporotic phenotype observed in our mice, despite normal serum levels of circulating PTH. Accordingly, it is clear from the observations of the present studies that the effectiveness of PTH 1-34 may be influenced by local PTHrP levels at the osteoblast microenvironment. We observed that the anabolic effect of PTH 1-34 is more profound in PTHrP^{+/-} mice in that not only does it equal but rather surpasses that observed in treated wild type animals. One skilled in the art may suggest that this observation may be due to the altered levels of signaling via the common type 1 PTH/PTHrP receptor. For example, normal concentrations of PTHrP in bone would tend to increase PTH/PTHrP receptor internalization and desensitization at the level of the osteoblast. However, osteoblast cell surface expression of PTH/PTHrP receptor, and hence its anabolic signaling, would be increased when PTHrP levels are decreased.

There is provided a transgenic non-human mammal heterozygous for disrupted PTHrP (i.e. PTHrP^{-/+}), where the disrupted portion is in exon 4 of PTHrP; preferably the mammal is a mouse; more preferably the mammal is used for studying effects of compounds re prevention/treatment of human disease; and additionally the mammal is used for studying bone development relating to the prevention/treatment of bone disease. In a preferred embodiment, the mammal is used to screen compounds/agents for therapeutic/prophylactic effects relating to preventing/treating/delaying bone disease; wherein bone disease is osteoporosis, osteomalacia, osteopenia, osteopetrosis, Paget's disease, or renal osteodystrophy. Accordingly the transgenic mammals provided are valuable models for the study of genetic osteoporosis; the transgenic mammals may be used to screen compounds for potentially identifying compounds that have an anabolic effect on bone. The model of the present invention has been confirmed as a valuable and reliable model for the study and detection of compounds affecting bone disease. More specifically, known bone anabolic compounds, namely PTH (1-34) which is analogous to PTH (1-84), PTHrP (1-36), PTHrP (1-139) and related analogs have illustrated the effectiveness of the transgenic mammals of the present invention for the described purposes. As previously described, the administration of exogenous PTH (1-34) in PTHrP^{-/+} mice vs. wild type mice via subcutaneous injection (40 ug/kg/day 6 times/week for 12 weeks) provided the results of FIG. 12. The parameters that were looked at were for the wild type vs. PTHrP^{-/+} mice are summarized in Table 1 below:

TABLE 1

| | wild type | PTHrP^{-/+} |
|---|---|---|
| Parameter | mice | mice* |
| BV/TV (˜bone volume) | 19% | 278% |
| Tb. N (trabecular number) | 24% | −4% |
| Tb. Th. (thickness) | 55% | 12% |
| connectivity | 306% | 37% |
| DA (degree of anisotropy) | 9% | −6% |
| Tb. Sp. (trabecular spacing) | −22% | 3% |
| SMI (structure model index) | −32% | −1% |

*PTHrP+/− mice (heterozygous for all cells)

The above summary of results confirms that the administration of PTH (1-34) in wild type mice did have a modest effect on all parameters; while profound anabolic changes were observed in PTHrP+/− mice (heterozygous for all cells). The above results, further confirm that the transgenic mammal of the present invention may be used to study and identify compounds affecting bone disease.

With the PTH^{-/-}/PTHrP^{-/+} double mutant mice (i.e. homozygous for disrupted PTH and heterozygous for disrupted PTHrP) it was observed that while PTH serves to maintain Ca homeostasis by restoring bone; locally produced PTHrP acts to promote bone formation. This observation was further confirmed by looking at the osteoporosis phenotype arising in PTHrP^{+/-} mice and PTHrPflox/flox crecol I mice; wherein the effectiveness of PTH (1-34) administration is influenced by local PTHrP levels at the osteoblast microenvironment. The anabolic effects are more profound in PTHrP^{-/+} mice. Accordingly, the studies of the present invention examined ‘osteo-compromised’ mice (for example, PTHrP^{-/+} mice in all cells, and PTHrP^{-/-} in osteoblast cells) and have shown the anabolic effects of PTH (1-34), which thereby confirms the ability of the transgenic mice model for the study of bone disease and for the use of the models as screening tools for detecting compounds or agents that may be potentially therapeutic or prophylactic or may delay the onset or progression of bone disease.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”.

The following examples are illustrative of various aspects of the invention, and do not limit the broad aspects of the invention as disclosed herein.

### EXAMPLES

### Example 1

### Analysis of PTHrP (+/−) Heterozygous Mice

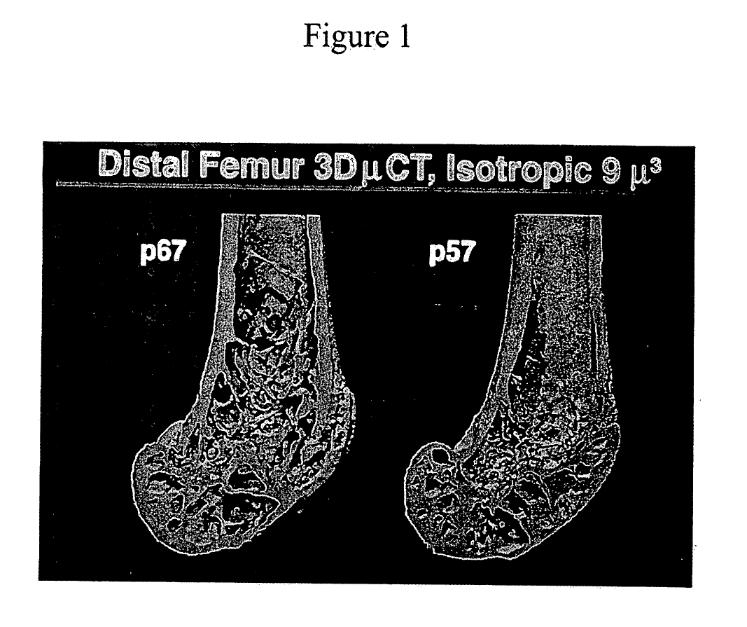

To assess the degree of bone loss in the PTHrP (+/−) animals, bones were removed from these animals and analyzed using microCT. The bones were scanned on a μCT 20 system (Scanco USA, Inc. Wayne, Pa.) at a resolution of 18 μm³. A set of images was obtained from each sample. Three-dimensional analysis was conducted on 10 manually selected volume of interest to calculate trabecular bone morphometric parameters. FIG. 1 is a representative picture from the normal (left specimen) and heterozygous mice (right specimen) showing again the diminished content of trabecular bone in the mutant animals.

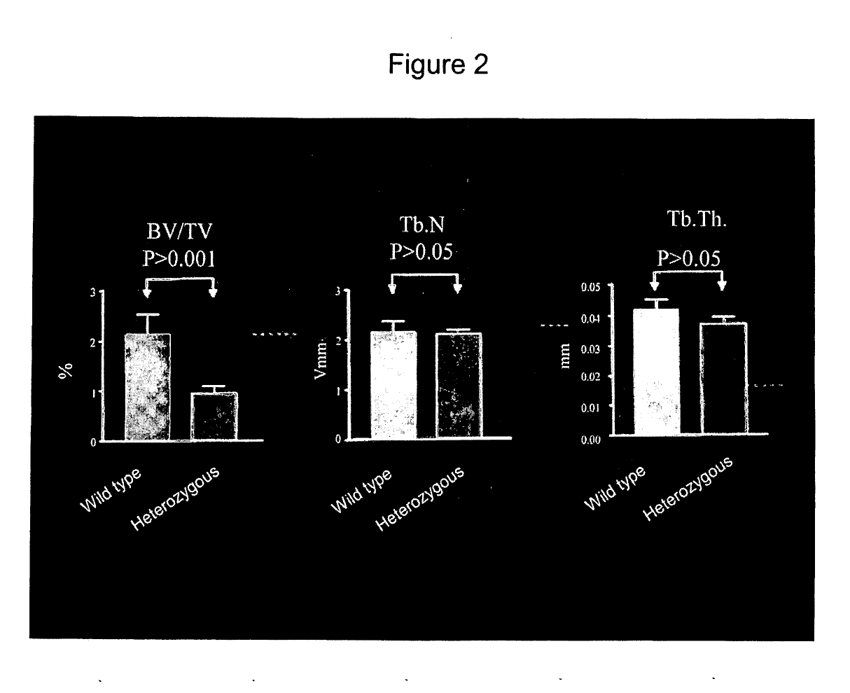

FIG. 2 shows the measured bone volume (BV/TV), analysed as described above, which is decreased by more than 50% in the mutant animals while the number (Tb.N) and thickness (Tb.Th.) of bone trabecules was not altered.

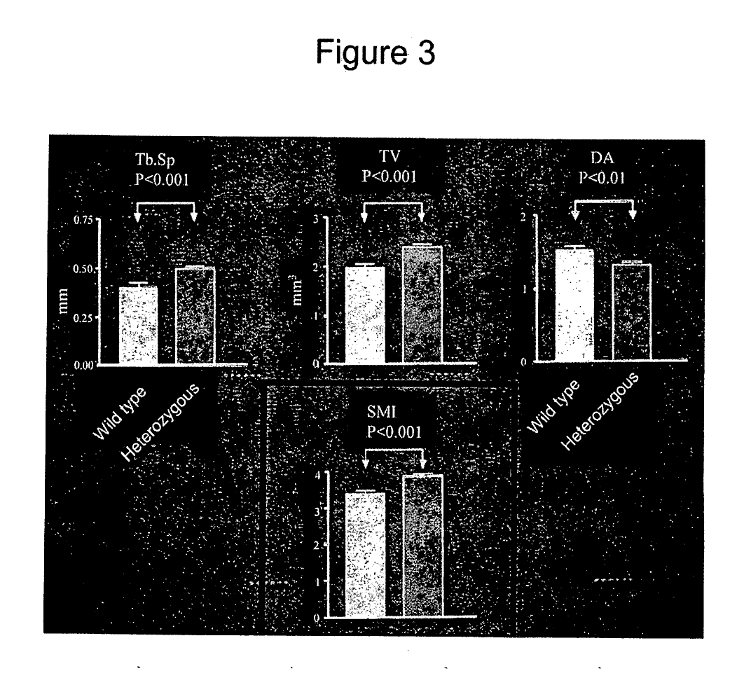

As shown in FIG. 3, the spacing between trabecules was significantly increased, while other parameters such as DA (degree of anisotropy) and SMI (Structure Model Index), features that describe the 3-D architecture of the bone, strongly suggested the presence of osteoporotic changes in the mutant mice.

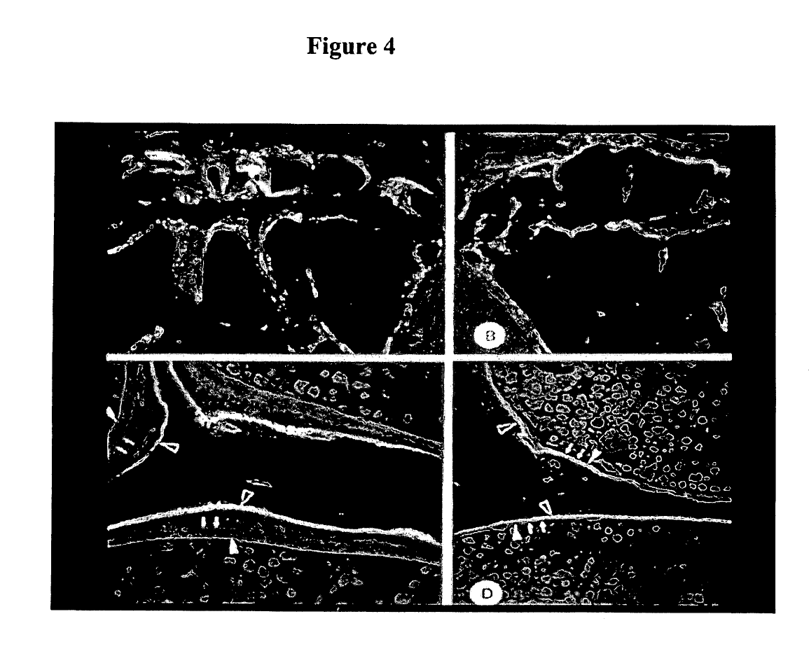

To ascertain the reason for the decreased amount of bone, mice were injected intraperitoneally with calcein (20 mg/kg body weight) and oxytetracycline (30 mg/kg body weight) and 4 days prior to sacrifice, respectively. One femur and one tibia per animal was removed and processed undecalcified in methylmethacrylate (a chemical that incorporates in newly deposited bone and gives off green fluorescent light), and 14 days later the same animals were injected with tetracycline (gives off orange red light). As shown in FIG. 4, the distance between the two lines (Panels C and D) can be used to calculate the rate of bone formation, which is markedly diminished in the mutant mice.

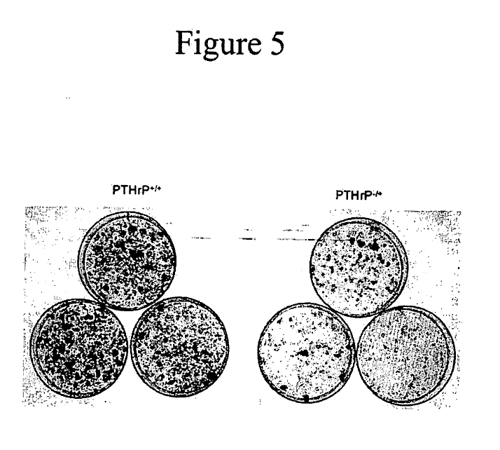

To evaluate the reason for the decreased bone formation in the PTHrP (+/−) mice, as shown in FIG. 5, bone marrow from normal (left) and mutant (right) animals was extracted, cultured and induced to differentiate into osteoblasts (bone forming cells). Bone marrow was flushed from both ends with 1 ml a-MEM medium and cell cultures prepared. Adherent cells were recovered and plated at a density of 104 cells/35 mm dish in a-MEM containing 10% fetal calf serum, antibiotics (100 ug/ml of pen G, 50 ug/ml of gentamycin, and 0.3 ug/ml of Fungizone), 50 ug/ml of ascorbic acid, 10-9 M dexamethasone and 10 mM Na—R-glycerophosphate to promote the formation of mineralized bone nodules. Medium was changed every 2-3 days, for 20 days. At the end of this period, cells were fixed and stained for alkaline phosphatase. Colonies were quantified using a dissecting microscope. Here, it is shown that the mutant marrow cannot differentiate into osteoblasts as readily as that of normal mice. Therefore, osteoblast differentiation is impaired.

### Example 2

### Selective Inactivation of PTHrP in Osteoblasts in Mice

In the heterozygous PTHrP (+/−) mice, the PTHrP gene is removed from every cell in the body, including osteoblasts and chondrocytes, the cartilage-forming cells. Since bone is derived from cartilage, it is possible that the observations were a reflection of improper cartilage formation per se rather than impaired bone formation. These observations point to a potential mechanism with the same end result, i.e. impaired bone formation in PTHrP heterozygotes.

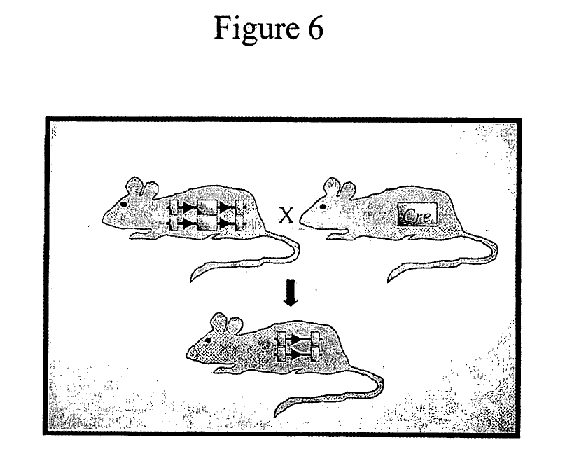

To rule out this possibility, mice were generated (shown schematically in FIG. 6) missing PTHrP only from osteoblasts using the Cre-LoxP system for which two mice are needed: one contains the PTHrP gene flanked by LoxP sequences and the second, is a transgenic mouse expressing Cre recombinase under an osteoblast-specific promoter (type 1 collagen) (He et al. (2001) Endocrinology 142(5):2070-7). When mated, the PTHrP gene is removed only from osteoblasts. All other cells will be normal. Specifically, mice carrying the site-specific recombinase gene (cre) under the control of the type I collagen (Col I) promoter were crossed with PTHrP floxed mice (PTHrPflox/flox) in which the exon 4 of both PTHrP alleles was flanked by loxP sites to generate mice with osteoblasts lacking PTHrP expression (PTHrPflox/flox crecol I).

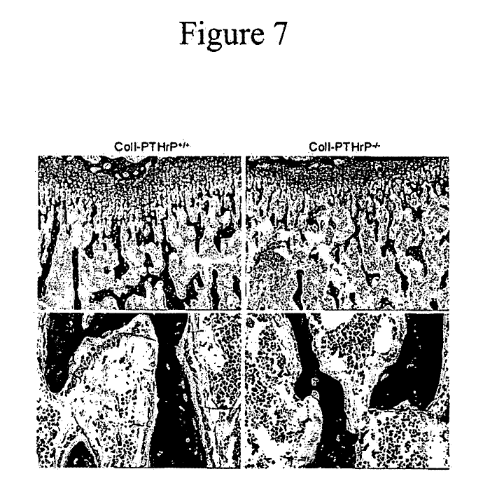

As shown in FIG. 7, osteoblasts developed normally in trabecular bone (as shown in the top panels). The trabecules (black color) are lined with functional osteoblasts. In contrast, mutant mice (lower panel) does not display a significant number of healthy osteoblasts. Therefore, absence of PTHrP from osteoblasts leads to poor osteoblast function and diminished bone formation.

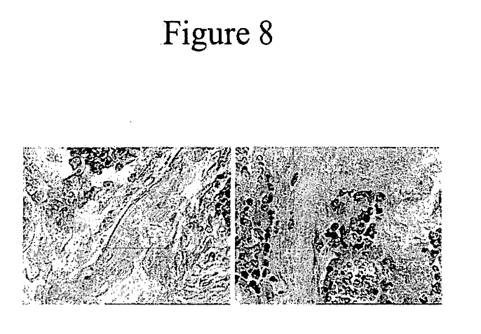

As shown in FIG. 8, osteoblasts missing PTHrP were not very evident in bone because they died by apoptosis. As shown in the right panel, staining of osteoblasts with a specific assay for apoptotic nuclei is very much evident in the mutant animals missing PTHrP (2 months of age) (red color, right panel) but not in the wild type specimens (left panel).

These findings indicate that PTHrP is critical for normal osteoblast development, since its absence leads to decreased osteoblast generation and early apoptotic death. The final result is decreased bone formation and the premature development of osteoporosis.

It was further determined that serum calcium, PTH and 1,25 dihydroxyvitamin D3 levels and parathyroid gland size were normal in 6 week PTHrPflox/flox crecol I mice as compared to the wild type (PTHrPflox/flox) control littermates. Bone density of femurs and tibiae on the other hand were found to be decreased by 6.33% and 9.00% in 6 week PTHrPflox/flox cre Col I mice, as compared to wild type mice as measured by PIXImus densitometer. Trabecular bone volume was also affected where femurs and tibiae were decreased by 33.33% and 35.50% respectively in newborn PTHrP^{flox/flox} cre^{col I} mice and 33.90% and 47.00% in 6 week PTHrP^{flox/flox} cre^{col I} mice, in comparison to their wild type littermates. Histomorphometric analysis revealed that osteoblast number and surface, osteoid thickness, trabecular thickness all were reduced significantly in 6 week PTHrP^{flox/flox} cre^{col I} mice compared to wild type mice.

This was further substantiated by immunohistochemistry as assessed by measurements of positive areas for type I collagen, osteocalcin, and osteopontin staining in the metaphysis of femurs of 6 week PTHrP^{-/flox} cre^{col I} mice compared to their wild type counterparts. Bone formation rates were also reduced as demonstrated by double calcein label histomorphometry when compared to 6 week wild type mice. Furthermore, osteoblast 30 number and surface were also reduced significantly in 6 week PTHrP^{-/flox}; cre^{col I} mice compared to wild type mice. These results therefore suggest that PTHrP exerts its anabolic action at least in part by an autocrine manner and may regulate or stimulate osteoblast formation and recruitment.

The above protocol was adapted to generate heterozygous osteoblast-specific PTHrP disrupted animals, in accordance with the present invention.

### Example 3

### Comparison of PTHrP Genetic Sequences Between Osteoporotic and Healthy Subjects

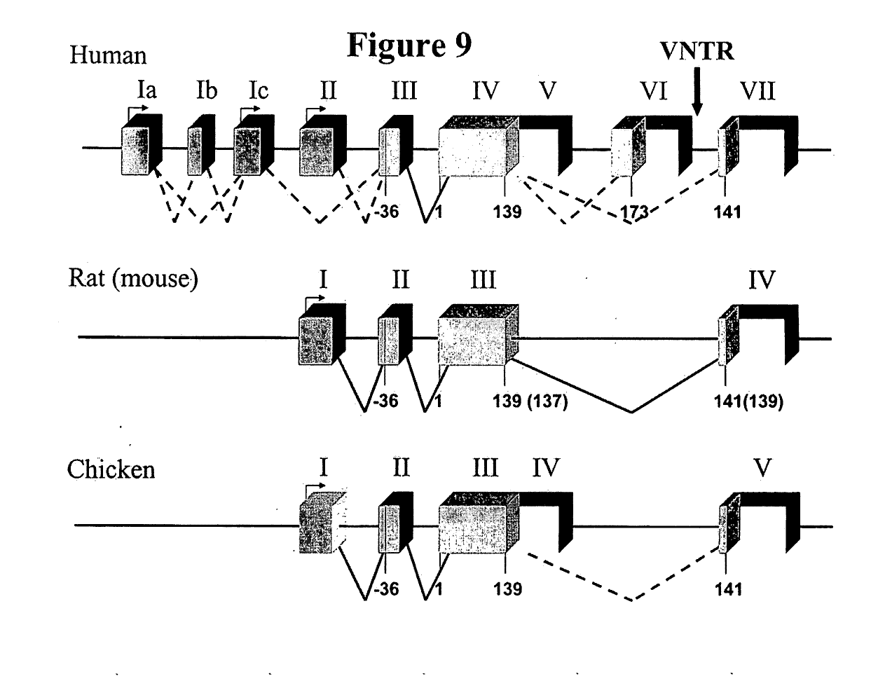

The human PTHrP gene structure is depicted among others in FIG. 9, and is further disclosed by Yasuda T. et al. in J Biol Chem. 1989 May 5; 264(13):7720-5). The arrow points to the region of DNA that encompasses a VNTR, a Variable Number of Tandem Repeats, that has been previously described but no functionality was ascribed to it (Pausova Z. et al. Genomics. 1993 17(1):243-4).

As illustrated in FIG. 10, the VNTR-containing sequence can be amplified from genomic DNA using PCR with the oligonucleotide primers for the regions underlined. The number of tandem repeats (G/ATATATATA)n gives rise to various lengths of amplified DNA depending on the number (n) of these repeats contained in an individual's DNA.

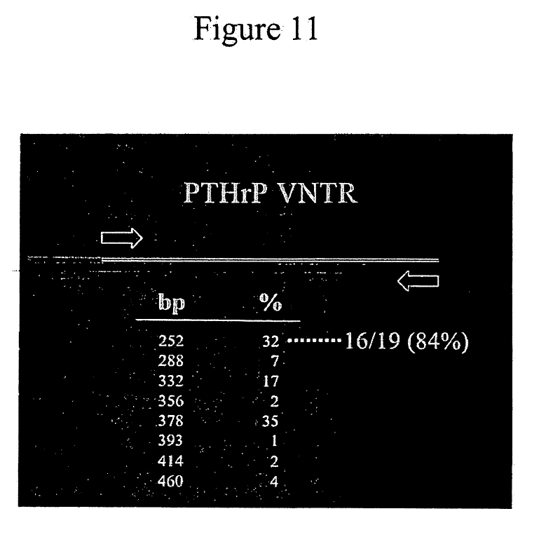

As shown in FIG. 11, the prevalence of the various VNTRs in the general population, ranges from 252 base pairs (bp) to 460 bp in length. Clearly, the 252 bp and the 378 bp VNTRs are the most common, although several less frequent ones are also indicated.

The PTHrP VNTR region in 19 osteoporotic male subjects was then examined. These patients had the diagnosis of idiopathic osteoporosis, i.e. likely due to a genetic etiology. As shown in FIG. 11, in this sample of patients, 16/19 (84%) had the 252 bp allele, a frequency much higher than that seen in the general population (32%), indicating that short VNTRs may be important in determining a higher risk for osteoporosis in a specific individual.

### Example 4

### PTHrP VNTR Genotyping Protocol

Below is provided a preferred PTHrP VNTR genotyping protocol in accordance with the present invention. The following example is illustrative of various aspects of the invention, and does not limit the broad aspects of the invention as disclosed herein.

Buffy Coat Preparation: In a preferred embodiment, blood samples were kept on ice immediately upon collection from patients and centrifuged at 1 800 rpm for 10 minutes at 4° C. The buffy coat was isolated and kept at −80° C. until DNA extraction was performed.

DNA Extraction: DNA extraction was preferably performed using the QIAamp DNA Blood Mini-Kit from QIAgen. Into 1.5 ml microcentrifuge tube, 27 μl QIAGEN Protease was added; a 200 μl sample was added to the microcentrifuge tube. Up to 200 μl of buffy coat was used; if the sample was less than 200 μl, the appropriate volume of PBS was added. Subsequently, 200 μl buffer AL was added to the sample; and mixed by pulse-vortexing for 15 seconds. The sample was incubated at 56° C. for 10 minutes. The 115 ml microcentrifuge tube comprising the test sample were briefly centrifuged to remove drops from the inside of the lid. 200 μl ethanol (100%) was added to the sample, and mixed again by pulse-vortexing for 15 seconds. After mixing briefly, the 1.5 ml microcentrifuge tube were centrifuged to remove drops from inside the lid. The mixture was carefully applied to the QIAamp spin column (in a 2 ml collection tube) without wetting the rim, the cap was closed, and centrifuged at 8 000 rpm for 1 minute. The QIAamp spinned column was placed in a clean 2 ml collection tube, and discarded the tube containing the filtrate. The QIAamp spin column was carefully opened and 500 μl Buffer AW1 was added without wetting the rim. The cap was closed and centrifuge at 8 000 rpm for 1 minute. The QIA amp spin column was placed in a clean 2 ml collection tube, and the collection tube containing the filtrate was discarded. The QIAamp spin column was carefully opened and 500 μl Buffer AW2 was added without wetting the rim. The cap was closed and the column centrifuge at full speed (14 000 rpm) for 3 minutes. The QIA amp spin column was placed in a new 2 ml collection tube and the collection tube with the filtrate was discarded; centrifuged at full speed for 1 minute. The QIAamp spin column was placed in a clean 1.5 ml microcentrifuge tube, and the collection tube containing the filtrate was discarded. The QIAamp spin column was carefully opened and 200 μl Buffer AE was added. The column was incubated at room temperature for 5 minutes; centrifuged for 1 minute. DNA samples were kept at 4° C. DNA was quantified using UV/VIS spectrophotometer at wavelengths of 260 and 280 nm.

PCR Amplification PCR amplification of the PTHRP VNTR was performed on a 25 μl volume using a negative control where no DNA was included in the reaction. A positive control consisting of a DNA sample from the experiment was also used as an internal standard. In a preferred protocol: the primer sequences used were:

Oligo Forward

(SEQ ID NO:6)

5′-GACCTAGTTCTGATTGTATCCTCTACC-3′
Oligo Reverse

(SEQ ID NO:7)

5′-GTTCCAGGCGTAAGAATTGACGAGTG-3′

The reagent concentration in each PCR reaction was:

100 ng genomic DNA

1× buffer

0.368 μM OligoF

0.368 μM OligoR

200 μM dNTP

1.5 unit Taq polymerase

The thermal cycle consisted of:

2 minutes at 94° C.

20 seconds at 94° C.

1 minute at 63° C.

30 seconds at 72° C.

7 minutes at 72° C.

Steps 2 to 3 were repeated 27 times

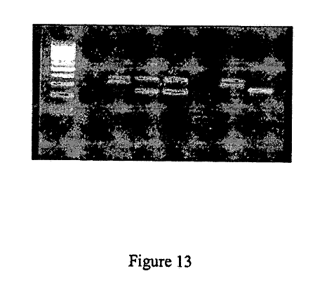

Agarose Gel Electrophoresis The amplified PTHrP VNTR was separated according to molecular size on a 2.0% agarose gel, stained with ethidium bromide and visualized under UV light. The Generuler 100 bp DNA ladder from Fermentas was used as a standard ladder. An aliquot of 3 μl of PCR product and 1 μl loading buffer was loaded onto the agarose gel. FIG. 13, provides an agarose gel of amplified PTHrP VNTR separated according to molecular size on a 2.0% agarose gel, stained with ethidium bromide and visualized under UV light; Generuler 100 bp DNA ladder from Fermentas was used as a standard ladder; an aliquot of 3 μl of PCR product and 1 μl loading buffer was loaded onto the agarose gel.

### Materials and Methods

Study Design

Patients presenting to the osteoporosis clinic with the finding of a low or normal bone mass density (BMD) were invited to enter the study and a consent form will be signed. The diagnosis of osteoporosis due to genetic etiology will be made upon the presentation of a positive family history and exclusion of secondary etiologies. Lumbar spine (L2-4), femoral neck, and trochanter T− (BMD compared to young adults) and Z− (BMD compared to age matched controls) scores will be recorded. Also baseline characteristics such as age, height, weight, calcium intake, 25 (OH) vitamin D levels, will be obtained. A blood sample (10 cc) will be collected from every patient. DNA will be isolated from the blood cells and assayed by Polymerase Chain Reaction (PCR) for the PTHrP VNTR. Amplified DNA fragments will then be examined for their length using polyacrylamide gel electrophoresis (PAGE).

Patient's confidentiality will be respected, under the limits of the law. If they wish, patients can terminate their participation in this study at any time. There will be no costs, nor payments, for patients participating in this study.

Exclusion Criteria

Patients with history of bone disease or those that are taking medications that affect bone turnover will be excluded from the study. Patients that are already being treated for osteoporosis will not be enrolled in the study.

Risks for the Patients

The risks related to this study are minimal. They are associated with taking blood and include pain, bruises, or, rarely, fainting.

Benefits for the Patients

The patients will be checked for osteoporosis and will receive appropriate follow up and treatment in case of a decrease in their BMD.

Relevance

The PTHrP VNTR genetic marker will be used as a diagnostic tool in the assessment of individuals at risk of developing osteoporosis and osteoporotic fractures. We already know that the BMD T-score value of −2.5, which is widely used as a treatment threshold for osteoporosis, identifies only a small proportion of individuals in the community who actually suffer fractures. Genetic markers of bone fragility or bone loss could be used prior to and alongside BMD measurements to help target early preventative therapies to those individuals who are at risk of fracture.

Statistical Analysis

This is an exploratory study of the predictive value of VNTR length on BMD. The length of VNTR in each patient will be recorded. As well, 2 markers of BMD will be recorded namely: Lumbar spine and femoral neck.

The statistical objective will be to show that there is a statistically significant relationship between VNTR length and two predictor variables of BMD. The outcome measures are:

Response:
Y=VNTR length, a continuous measure

Predictors:
X1=Lumbar Spine,
X2=Femoral neck,
X3 Trochanter,
where all three are continuous variables.

This relationship will be controlled for baseline characteristics such as age, height, weight, calcium intake and 25 (OH) vitamin D levels. Sex will be another control variable but with a nesting of the level of osteoporosis.

There will be a total of 80 participants in this study: two groups of males with 20 participants per group: Normal and Osteoporotic; two groups of premenopausal females with 20 participants per group: Normal and Osteoporotic.

The following analysis of covariance analyses will be performed:
Y=X1+X2+X3+age+height+weight+calcium+vitamin+Sex+Osteoporosis level (sex)

All baseline characteristics that are not significant will be dropped from the analysis and subsequent conclusions based on the reduced model. All analyses will be performed in SAS version 8.12. Statistical significance of the regression coefficients of X1, X2, and X3 will be indicative of probable predictive value, to be confirmed in further studies.

### Example 5

### Development of an Osteoblast-Specific Oligonucleotide

We have recently shown that the following oligonucleotide [SEQ ID NO: 1]:

5′-TATATACGTATATATATATACGTATATATATACGTA-3′

comprising the GTATATATA sequence of the VNTR is bound by nuclear proteins when the oligonucleotide was incubated with cell nuclear extracts. Specifically, when we incubated the VNTR oligonucleotide with COS cell nuclear extracts (non-osteogenic cell line) there was no specific binding of any nuclear proteins. However, when we incubated the VNTR oligonucleotide with ROS cell nuclear extracts (osteogenic cell line) there was specific binding of many nuclear proteins.

The precise nature of these protein(s) remains to be determined. However, this particular region is critical for PTHrP expression and the protein(s) that are binding to the oligonucleotide sequence [SEQ ID NO: 1] could lead to the discovery of novel therapeutics and diagnostic markers of bone disease.

The invention also includes nucleic acids that hybridize under moderately stringent, or preferably stringent hybridization conditions, to all or a portion of the oligonucleotide sequence represented by [SEQ ID NO: 1] or its complement. Stringent conditions are sequence dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993) (Elsevier Science, Inc., New York). Generally, stringent conditions are selected to be about 5-10° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization.

Exemplary stringent hybridization conditions can be as following: 50% formamide, 5×SSC, and 1% SDS, incubating at 42° C. or, 5×SSC, 1% SDS, incubating at 65° C., with wash in 0.2×SSC, and 0.1% SDS at 65° C. For the purpose of the invention, suitable “moderately stringent conditions” include, for example, pre-washing in a solution of 5×SSC, 0.5% SDS, 1.0 mM EDTA (pH 8.0), hybridizing at 50° C.-65° C., 5×SSC overnight, followed by washing twice at 65° C. for 20 minutes with each of 0.5× and 0.2×SSC (containing 0.1% SDS). Such hybridizing DNA sequences are also within the scope of this invention.

### Example 6

### Comparison of PTHrP Genetic Sequences Between Osteoporotic and Healthy Subjects: Second Clinical Study

The following results confirm the findings of the first clinical study described above in Example, 3, which was conducted on 19 male osteoporotic subjects and which showed a significant association between a certain variation in the Parathyroid Hormone Related Peptide (PTHrP) gene and the age matched Bone Mineral Density (BMD Z score) values at hip or spine. The second clinical study involved a total of 11 healthy or osteoporotic male subjects and was undertaken to not only confirm the findings of the first study in osteoporotic males, but also to look at the trends in healthy males.

This second clinical study was conducted at the Jewish General Hospital, a McGill University teaching hospital based in Montreal, Quebec, Canada. The primary objective of this study was to study the association of the frequency of the 252 bp allele within the VNTR region of the PTHrP gene with the BMD Z scores at the hip or spine. The experiment was conducted substantially identically to the first clinical study presented in Example 3.

Results:

The association between the frequency of the 252 bp allele in the VNTR region of the PTHrP gene was studied as a function of Bone Mineral Density (BMD) in at least one of the skeletal sites: hip or spine. Age matched BMD Z scores were used for the purpose of data analysis. The results of the study are shown in Table 2.

TABLE 2
Results of Second Clinical Study

| | Number | | | | |
|---|---|---|---|---|---|
| | of | | | | |
| | healthy | Number of | | | |
| | control | osteoporotic | | Chi | |
| | subjects | subjects | E | square | P |
| Total Males | 6 | 5 | — | — | — |
| in 2^{nd} | | | | | |
| study | | | | | |
| Males with | 1 | 3 | 1.0 | 4.00 | 0.017 |
| at least | | | | | |
| one 252 bp | | | | | |
| allele in | | | | | |
| 2^{nd} study | | | | | |
| Combined | 6 | 24 | — | — | — |
| Total males | | | | | |
| in 1^{st} and | | | | | |
| 2^{nd} study | | | | | |
| Combined | 1 | 19 | 4.0 | 56.25 | <0.0001 |
| total males | | | | | |
| with at | | | | | |
| least one | | | | | |
| 252 bp | | | | | |
| allele in | | | | | |
| 1^{st} and 2^{nd} | | | | | |
| study | | | | | |

A statistically significant trend was seen in males in the second study as well as the combined first and second studies. The first clinical study conducted on 19 male osteoporotic subjects had shown that 16 out of 19 (84%) male subjects had at least one copy of the 252 bp allele in the VNTR region of their PTHrP gene. However, there were no healthy control males in the first study and it was not possible to calculate the P values in order to evaluate the statistical significance of a potential association between the frequency of the 252 bp allele with the BMD Z scores. In the second study, only 1 out of 6 (17%) healthy males were found to have at least one copy of the 252 bp allele in the VNTR region of their PTHrP gene, whereas, 3 out of 5 (60%) male osteoporotics were found to have at least one copy of the 252 bp allele in the VNTR region of their PTHrP gene. In the combined first and second study on males, 1 out of 6 (17%) healthy males were found to have at least one copy of the 252 bp allele in the VNTR region of their PTHrP gene, whereas, 19 out of 24 (79%) male osteoporotics were found to have at least one copy of the 252 bp allele in the VNTR region of their PTHrP gene. The results of the second study confirm the findings of the first study, specifically the fact that osteoporotic males tend to have a higher frequency of the 252 bp allele in their PTHrP VNTR compared to healthy males, in a statistically significant fashion.

As can be seen from these results, the number of males in the second clinical study with at least one 252 bp allele is significantly higher in osteoporotic males than in the healthy control population, with a P value of 0.017. When these results are taken together with the study reported in Example 3 (the first study), the result is even more pronounced as seen in the bottom row of Table 2 (P<0.0001).

The embodiments) of the invention described above is(are) intended to be exemplary only. The scope of the invention is therefore intended to be limited solely by the scope of the appended claims.

I/We claim:

1. A method of diagnosing a male human for the susceptibility or predisposition to osteoporosis or osteopenia, said method comprising:
a) amplification of a variable number tandem repeat (VNTR) region within an intronic region between exons VI and VII of a parathyroid hormone related peptide (PTHrP) gene in a biological sample from said human using primers corresponding to the sequences of SEQ ID NO: 6 and SEQ ID NO: 7 or complements thereof;
b) measuring the length of the amplified VNTR region of said human; and
c) diagnosing said human based on the results of step (b), wherein the presence of a VNTR region 252 base pairs in length is correlated with a diagnosis for susceptibility or predisposition to osteoporosis or osteopenia.

2. The method of claim 43, where said biological sample is a biological fluid or tissue comprising isolatable genomic DNA.


SEQUENCE LISTING (7 sequences)

SEQ ID NO: 1 - DNA, 36 bp, Artificial Sequence
  Description of Artificial Sequence oligonucleotide
tatatacgtatatatatatacgtatatatatacgta

SEQ ID NO: 2 - DNA, 9 bp, Homo Sapiens
  VNTR sequence; R is G or A
rtatatata

SEQ ID NO: 3 - DNA, 9 bp, Homo Sapiens
  VNTR sequence
gtatatata

SEQ ID NO: 4 - DNA, 9 bp, Homo sapiens
  VNTR sequence
atatatata

SEQ ID NO: 5 - DNA, 376 bp, Homo sapiens
  Sequence of 252 bp allele of PTHrP gene VNTR region
gacctagttctgattgtatcctctacctatcatctatctatttatatctatctatctatctagggggatatgtttctatgaggcaaaaagtatatatatacgtatatatatatatatatatacacgtatatatatatacgtatatatatatacgtatatatatgcgtatatatatatacgtatatatatatacgtatatatatacgtatatatatatacgtatatatatacgtatatatatatacgtatatatatgagttataaaaagaggtataaataacttttgacggattttatttatttcaaatttgcaatgttctttaagtatctggagaattgatgaaaattaacactcgtcaattcttacgcctggaac

SEQ ID NO: 6 - DNA, 27 bp, Homo sapiens
  Forward PTHrP amplification primer
gacctagttctgattgtatcctctacc

SEQ ID NO: 7 - DNA, 26 bp, Homo sapiens
  Reverse PTHrp amplification primer
gttccaggcgtaagaattgacgagtg